(19) United States
(12) Patent Application Publication
(10) Pub. No.: US 2023/0295683 A1
(43) Pub. Date: Sep. 21, 2023

(54) ISOLATION OF FETAL CELLS

(71) Applicant: Luna Genetics, Inc., Houston, TX (US)

(72) Inventors: Arthur L. BEAUDET, Houston, TX (US); Mason OUREN, Houston, TX (US); Michelle BELLAIR, Houston, TX (US); Jaeweon KIM, Houston, TX (US)

(21) Appl. No.: 18/157,007

(22) Filed: Jan. 19, 2023

Related U.S. Application Data
(60) Provisional application No. 63/301,158, filed on Jan. 20, 2022.

(51) Int. Cl. C12Q 1/24; C12Q 1/6881

(52) U.S. Cl. CPC C12Q 1/24; C12Q 1/6881; C12Q 2600/124

(57) ABSTRACT

This disclosure generally relates to isolation of fetal cells from biological samples. Methods of using the enriched fetal cells for detecting genetic or epigenetic abnormalities or variations are also provided herein.

### RELATED APPLICATION

This application claims priority to U.S. Provisional Patent Application Ser. No. 63/301,158, filed on Jan. 20, 2022, which is incorporated herein by reference in its entirety for all purpose.

### INCORPORATION OF THE SEQUENCE LISTING

This application contains a Sequence Listing, which is hereby incorporated by reference in its entirety. The accompanying Sequence Listing text file, named “2023-04-27 Sequence_Listing_ST26 052074-502001US.xml” was created on Apr. 27, 2023 and is 2.5 KB.

### FIELD OF THE DISCLOSURE

This disclosure generally relates to isolation of fetal cells from biological samples, such as, but not limited to, maternal biological samples. The isolated fetal cells can be used to identify genetic or epigenetic abnormalities or variations in the fetus.

### BACKGROUND

Currently, the methods for directly testing for genetic or epigenetic abnormalities in the fetal cells are amniocentesis and chorionic villus sampling (CVS) and percutaneous umbilical blood sampling (PUBS). In amniocentesis, a sample of amniotic fluid is drawn by needle, and free-floating fetal cells in the fluid are examined. It is usually performed in the second trimester, between weeks 15 and 20 although also possible through term. Although it provides a definitive source of fetal DNA, the procedure carries a risk of miscarriage (L. J. Salomon et al., Ultrasound Obstet Gynecol (2019) 54(4):442-51; J. Beta et al., Minerva Ginecol (2018) 70(2):215-19; R. Akolekar et al., Ultrasound Obstet Gynecol (2015) 45:16-26. In CVS, a tissue sample is taken from the placenta and examined. Although this tissue derives from the embryo, it sometimes differs from the fetus due to confined placental mosaicism (CPM), in which some of the placental cells are abnormal and other placenta cell are normal (about 1-2% of pregnancies). CVS carries a higher risk of complications (estimated at 1-2%), but can be performed earlier than amniocentesis, usually at 10-12 weeks gestation.

Cell-free fetal DNA (cffDNA) non-invasive prenatal testing (NIPT) relies on the detection and characterization of extracellular fetal DNA circulating in maternal blood. Cell-free fetal DNA (cffDNA) constitutes about 5-20% of the total cell-free DNA in a pregnant woman and derives from trophoblasts (placental cells). The cffDNA itself is found in the form of fragments, about 200 base pairs (bp) in length. These characteristics limit the accuracy and utility of cffDNA NIPT. There is a more recent report of longer fetal DNA molecules in maternal plasma, but the clinical utility of this material is not yet clear (S. C. Y. Yu et al., Proc Natl Acad Sci (2021) 118:e2114937118).

In contrast to CVS and amniocentesis, cell-based NIPT has low or no risk of complications. The primary challenge for cell-based NIPT is that the target cells are exceedingly rare at 1-2 cells/mL maternal blood (K. Krabchi et al., Clin Genet (2001) 60:145-50). Thus, there remains a medical need to develop methods of cell-based NIPT that are safe, accurate, and can be performed at a gestational time earlier than amniocentesis.

### SUMMARY

In some embodiments, the present disclosure provides a method of isolating fetal cells from a biological sample, said method comprising the steps of: (a) contacting the biological sample with one or more binding agents directed against at least one of the group consisting of a fetal cell marker, a maternal cell marker, and a nuclear marker, (b) sorting the fetal cells of the biological sample based on detection of the one or more binding agents bound to said at least one nuclear marker, a fetal cell marker, or a maternal cell marker, wherein the sorting is performed using a microfluidic cell separator, microbubbles-based cell sorting device, fluorescence activated cell sorting (FACS) device or magnetic activated cell sorting (MACS) device; and (c) determining the presence of the fetal cells as those bound by the binding agents from step (b). The biological sample may be obtained from the pregnant woman at a gestational age of less than about 17 weeks. The gestational age may be between from about 5 weeks and about 15 weeks. The one or more binding agents may be a nucleotide probe. The nucleotide probe may be an RNA probe, a DNA probe, a GNA probe or an LNA probe. The nucleotide probe may be an RNA probe. The nucleotide probe may be a DNA probe. The nucleotide probe may comprise at least two target probes and a probe system. The at least two target probes may be capable of hybridizing to non-overlapping sequences in fetal mRNA. The at least two target probes may be capable of hybridizing to the probe system. Each of the at least two target probes may comprise a first polynucleotide sequence F-1 that is complementary to a polynucleotide sequence in fetal mRNA and a second polynucleotide sequence F-2 that is complementary to a polynucleotide sequence in the probe system. The one or more binding agents may be an antibody or a fragment thereof.

The fetal cells may be sorted by a microfluidic cell separator. The microfluidic cell separator may sort the fetal cells at a pressure less than about 2 psi. The microfluidic cell separator may comprise a sheath fluid. The sheath fluid may have a flow rate of from about 5 mL/h to about 8 mL/h. The microfluidic cell separator may comprise a droplet size of from about 0.5 μl to about 2 μL. The microfluidic cell separator may have a processing speed of from about 2 to about 50,000 cells/second. The fetal cells may be sorted by fluorescence activated cell sorting (FACS) device. The fetal cells may be further isolated using a single-cell picking device.

The one or more binding agents may be directed against a fetal cell marker. The fetal cell marker may be a fetal cell nucleic acid marker. The fetal cell nucleic acid marker may be XIST, TTTY15, RPS4Y1, KRT7, EPCAM, HLA-G, ENG, or βHCG. The fetal cell marker may be a fetal cell surface marker. The fetal cell marker may be a fetal epithelial cell marker or a fetal endothelial cell marker. The fetal cell marker may be a fetal epithelial cell marker and wherein the fetal epithelial cell marker is cytokeratin, ALCAM/CD166, Aminopeptidase N/ANPEP, CD13, Alanyl Aminopeptidase, Basal Bodies of Cilia (LhS28), Basal Cell Cytokeratin, beta-Crystallin, beta-Defensin 2, beta-Defensin 3, Bmi-1 oncoprotein, BRCA1, BTEB1, Calcitonin Gene-Related Peptide (CGRP), Calcyclin, Carcinoembryonic Antigen (CEA), Cathepsin E (CaE), Caveolin-1, CD138 (Syndecan-1), CD151, CD46, Clara cell-specific protein, Connexin-43 (Cx43), Cornulin, CRNN, C1ORF10, SEP53, Cystatin C, Desmin, Desmocollin 2, Desmocollin 3, E-Cadherin, Epithelial Antigen antibody (Ber-EP4), Epithelial Membrane Antigen (EMA, MUC-1, CA 15-3, CD227), Epithelial Sodium Channel-α, Epithelial Sodium Channel-β, Epithelial Sodium Channel-γ, Epithelial Sodium Channel-δ, Epithelium specific antigen (EP-CAM, ESA) (AUA1), Epithelium/endothelial cells [PCX, Podocalyxin], Exo-1 (Pa-G14), EZH2, Ezrin, Fas Ligand/TNFSF6, Fibrinogen (1F3), Foxal, GABRP, Galectin-3, GGT (gamma-glutamyl transpeptidase), Glutamine Synthetase, Heat Shock Protein 27 [HSP27], HLA-DR, Lactoferrin, LAMP-1 (lysosomal-associated membrane protein 1), MMR, NCAM-L1 (neural cell adhesion molecule L1), Nectin-2/CD112, Normal Epithelial Cell Specific-1 (NES1)/kallikrein-10, NSE (neuron-specific enolase), Ovarian Cancer Antigen [CA125], P2X7, p63, P-Cadherin, pIgR, Prominin-1 (CD133), Prostasin/Prss8, Prostate Specific Antigen [PSA], Prostatic Binding Protein (PBP), Protein Gene Product 9.5 (PGP 9.5), PSCA (Prostate stem cell antigen), Rab13, RAGE, Rex-1 (zinc-finger protein-42, Zfp42), Secretory Component (SC), Sucrase-isomaltase (SI), Surfactant Protein A, Surfactant Protein B, Surfactant protein C (SPC), Surfactant Protein D, Survivin, TfR (Transferrin Receptor), Transthyretin, UGRP1/SCGB3A2, VAT-1, or Vimentin. The fetal epithelial cell marker may be cytokeratin. The cytokeratin may be Human Cytokeratin 1 (CK1), Human Cytokeratin 2 (CK2), Human Cytokeratin 3 (CK3), Human Cytokeratin 4 (CK4), Human Cytokeratin 5 (CK5), Human Cytokeratin 6 (CK6), Human Cytokeratin 7 (CK7), Human Cytokeratin 8 (CK8), Human Cytokeratin 9 (CK9), Human Cytokeratin 10 (CK10), Human Cytokeratin 13 (CK13), Human Cytokeratin 14 (CK14), Human Cytokeratin 15 (CK15), Human Cytokeratin 16 (CK16), Human Cytokeratin 17 (CK17), Human Cytokeratin 18 (CK18), or Human Cytokeratin 19 (CK19).

The fetal cell marker may be a fetal endothelial cell marker. The fetal endothelial cell marker may be CD146, CD141, ACE/CD143, C1qR1/CD93, VE-Cadherin, CC Chemokine Receptor D6, CD31/PECAM-1, CD34, CD36/SR-B3, CD144, CD151, CD160, CD300g/Nepmucin, CL-K1/COLEC11, CL-P1/COLEC12, Coagulation Factor III/Tissue Factor, DC-SIGNR/CD299, DCBLD2/ESDN, ECSCR, EMMPRIN/CD147, Endoglin/CD105, Endomucin, Endosialin/CD248, EPCR, Erythropoietin R, ESAM, FABPS/E-FABP, FABP6, ICAM-1/CD54, ICAM-2/CD102, IL-1 RI, IL-13 R alpha 1, Integrin alpha 4/CD49d, Integrin alpha 4 beta 1, Integrin alpha 4 beta 7/LPAM-1, Integrin beta 2/CD18, KLF4, LYVE-1, MCAM/CD146, Nectin-2/CD112, PD-ECGF/Thymidine Phosphorylase, Podocalyxin, Podoplanin, S1P1/EDG-1, S1P2/EDG-5, S1P3/EDG-3, S1P4/EDG-6, S1P5/EDG-8, E-Selectin/CD62E, E-Selectin (CD62E)/P-Selectin (CD62P), P-Selectin/CD62P, SLAM/CD150, Stabilin-1, Stabilin-2, TEM7/PLXDC1, TEM8/ANTXR1, Thrombomodulin/BDCA-3, THSD1, THSD7A, Tie-2, TNF RI/TNFRSF1A, TNF RII/TNFRSF1B, TRA-1-85/CD147, TRAIL R2/TNFRSF10B, TRAILR1/TNFRSF10A, VCAM-1/CD106, VE-Statin, VEGFR1/Flt-1, VEGFR2/KDR/Flk-1, VEGFR3/Flt-4, VGSQ, or vWF-A2. The fetal endothelial cell marker may be Endoglin/CD105. The one or more binding agents may be directed against a maternal cell marker. The maternal cell marker may be CD45.

The biological sample comprises a population of cells, wherein the population of cells may comprise at least one of a maternal cell and a fetal cell. The fetal cell may be present and wherein the fetal cells is a fetal trophoblast cell. The fetal cell may be a male fetal cell. The fetal cell may be a female fetal cell. The biological sample may be a blood sample or a cervical secretion sample. The biological sample may comprise a cellular fraction obtained from the blood sample. The one or more binding agents may be bound to a label. The label may be bound covalently or non-covalently to the one or more binding agents. The label may be a dye, a radiolabel, a hapten, a luminogenic, a phosphorescent or a fluorogenic moiety, or a mass tag. The label may be a dye and wherein the dye is a fluorescent dye.

The fluorescent dye may be a xanthene dye, a coumarin dye, a pyrene dye or a cyanine dye. The fluorescent dye may be Indo-1, Ca saturated, Indo-1 Ca2+, Cascade Blue BSA pH 7.0, Cascade Blue, LysoTracker Blue, Alexa 405, LysoSensor Blue pH 5.0, LysoSensor Blue, DyLight 405, DyLight 350, BFP (Blue Fluorescent Protein), Alexa 350, 7-Amino-4-methylcoumarin pH 7.0, Amino Coumarin, AMCA conjugate, Coumarin, 7-Hydroxy-4-methylcoumarin, 7-Hydroxy-4-methylcoumarin pH 9.0, 6,8-Difluoro-7-hydroxy-4-methylcoumarin pH 9.0, Hoechst 33342, Pacific Blue, Hoechst 33258, Hoechst 33258-DNA, Pacific Blue antibody conjugate pH 8.0, PO-PRO-1, PO-PRO-1-DNA, POPO-1, POPO-1-DNA, DAPI-DNA, DAPI, Marina Blue, SYTOX Blue-DNA, CFP (Cyan Fluorescent Protein), eCFP (Enhanced Cyan Fluorescent Protein), 1-Anilinonaphthalene-8-sulfonic acid (1,8-ANS), Indo-1, Ca free, 1,8-ANS (1-Anilinonaphthalene-8-sulfonic acid), BO-PRO-1-DNA, BOPRO-1, BOBO-1-DNA, SYTO 45-DNA, evoglow-Pp1, evoglow-Bs1, evoglow-Bs2, Auramine O, DiO, LysoSensor Green pH 5.0, Cy 2, LysoSensor Green, Fura-2, high Ca, Fura-2 Ca2+sup>, SYTO 13-DNA, YO-PRO-1-DNA, YOYO-1-DNA, eGFP (Enhanced Green Fluorescent Protein), LysoTracker Green, GFP (S65T), BODIPY FL, Sapphire, BODIPY FL conjugate, MitoTracker Green, MitoTracker Green FM, Fluorescein 0.1 M NaOH, Calcein pH 9.0, Fluorescein pH 9.0, Calcein, Fura-2, no Ca, Fluo-4, FDA, DTAF, Fluorescein, Fluorescein antibody conjugate pH 8.0, CFDA, FITC, Alexa Fluor 488 hydrazide-water, DyLight 488, 5-FAM pH 9.0, FITC antibody conjugate pH 8.0, Alexa 488, Rhodamine 110, Rhodamine 110 pH 7.0, Acridine Orange, Alexa Fluor 488 antibody conjugate pH 8.0, BCECF pH 5.5, PicoGreendsDNA quantitation reagent, SYBR Green I, Rhodaminen Green pH 7.0, CyQUANT GR-DNA, NeuroTrace 500/525, green fluorescent Nissl stain-RNA, DansylCadaverine, Rhodol Green antibody conjugate pH 8.0, Fluoro-Emerald, Nissl, Fluorescein dextran pH 8.0, Rhodamine Green, 5-(and-6)-Carboxy-2′,7′-dichlorofluorescein pH 9.0, DansylCadaverine, eYFP (Enhanced Yellow Fluorescent Protein), Oregon Green 488, Oregon Green 488 antibody conjugate pH 8.0, Fluo-3, BCECF pH 9.0, SBFI-Na+, Fluo-3 Ca2+, Rhodamine 123, FlAsH, Calcium Green-1 Ca2+, Magnesium Green, DM-NERF pH 4.0, Calcium Green, Citrine, LysoSensor Yellow pH 9.0, TO-PRO-1-DNA, Magnesium Green Mg2+, Sodium Green Na+, TOTO-1-DNA, Oregon Green 514, Oregon Green 514 antibody conjugate pH 8.0, NBD-X, DM-NERF pH 7.0, NBD-X, CI-NERF pH 6.0, Alexa 430, Alexa Fluor 430 antibody conjugate pH 7.2, CI-NERF pH 2.5, Lucifer Yellow, CH, LysoSensor Yellow pH 3.0, 6-TET, SE pH 9.0, Eosin antibody conjugate pH 8.0, Eosin, 6-Carboxyrhodamine 6G pH 7.0, 6-Carboxyrhodamine 6G, hydrochloride, Bodipy R6G SE, BODIPY R6G, 6 JOE, Cascade Yellow antibody conjugate pH 8.0, Cascade Yellow, mBanana, Alexa Fluor 532 antibody conjugate pH 7.2, Alexa 532, Erythrosin-5-isothiocyanate pH 9.0, 6-HEX, SE pH 9.0, mOrange, mHoneydew, Cy 3, Rhodamine B, DiI, 5-TAMRA-MeOH, Alexa 555, Alexa Fluor 555 antibody conjugate pH 7.2, DyLight 549, BODIPY TMR-X, SE, BODIPY TMR-X, PO-PRO-3-DNA, PO-PRO-3, Rhodamine, Bodipy TMR-X conjugate, POPO-3, Alexa 546, BODIPY TMR-X antibody conjugate pH 7.2, Calcium Orange Ca2+, TRITC, Calcium Orange, Rhodaminephalloidin pH 7.0, MitoTracker Orange, MitoTracker Orange, Phycoerythrin, Magnesium Orange, R-Phycoerythrin pH 7.5, 5-TAMRA pH 7.0, 5-TAMRA, Rhod-2, FM 1-43, Rhod-2 Ca2+, Tetramethylrhodamine antibody conjugate pH 8.0, FM 1-43 lipid, LOLO-1-DNA, dTomato, DsRed, Dapoxyl (2-aminoethyl) sulfonamide, Tetramethylrhodamine dextran pH 7.0, Fluor-Ruby, Resorufin, Resorufin pH 9.0, mTangerine, LysoTracker Red, Lissaminerhodamine, Cy 3.5, Rhodamine Red-X antibody conjugate pH 8.0, Sulforhodamine 101, JC-1 pH 8.2, JC-1, mStrawberry, MitoTracker Red, MitoTracker Red, X-Rhod-1 Ca2+, Alexa Fluor 568 antibody conjugate pH 7.2, Alexa 568, 5-ROX pH 7.0, 5-ROX (5-Carboxy-X-rhodamine, triethylammonium salt), BO-PRO-3-DNA, BOPRO-3, BOBO-3-DNA, Ethidium Bromide, ReAsH, Calcium Crimson, Calcium Crimson Ca2+, mRFP, mCherry, Texas Red-X antibody conjugate pH 7.2, HcRed, DyLight 594, Ethidium homodimer-1-DNA, Ethidiumhomodimer, Propidium Iodide, SYPRO Ruby, Propidium Iodide-DNA, Alexa 594, BODIPY TR-X, SE, BODIPY TR-X, BODIPY TR-X phallacidin pH 7.0, Alexa Fluor 610 R-phycoerythrin streptavidin pH 7.2, YO-PRO-3-DNA, Di-8 ANEPPS, Di-8-ANEPPS-lipid, YOYO-3-DNA, Nile Red-lipid, Nile Red, DyLight 633, mPlum, TO-PRO-3-DNA, DDAO pH 9.0, Fura Red, high Ca, Allophycocyanin pH 7.5, APC (allophycocyanin), Nile Blue, TOTO-3-DNA, Cy 5, BODIPY 650/665-X, Alexa Fluor 647 R-phycoerythrin streptavidin pH 7.2, DyLight 649, Alexa Fluor 647 antibody conjugate pH 7.2, Alexa 647, Fura Red Ca2+, Atto 647, Fura Red, low Ca, Carboxynaphthofluorescein pH 10.0, Alexa 660, Alexa Fluor 660 antibody conjugate pH 7.2, Cy 5.5, Alexa Fluor 680 antibody conjugate pH 7.2, Alexa 680, DyLight 680, Alexa Fluor 700 antibody conjugate pH 7.2, Alexa 700, FM 4-64, 2% CHAPS, or FM 4-64.

In some embodiments, the present disclosure provides a method of detecting fetal cells from a biological sample, said method comprising the steps of: (a) contacting the biological sample with one or more binding agents directed against at least one of the group consisting of a nuclear marker, a fetal cell marker, or a maternal cell marker, (b) visualizing the cells of the biological sample based on detection of the one or more binding agents bound to said at least one nuclear marker, a fetal cell marker, or a maternal cell marker; and (c) determining the presence of fetal cells as those bound by the binding agents from step (b). The cells may be visualized under a microscope or laser.

In some embodiments, the present disclosure provides a method for genotyping a fetus, the method comprising: (a) isolating fetal cells according to the methods of any one of claims 1 to 46; (b) lysing the fetal cells to obtain the fetal nucleic acid; (c) amplifying the fetal cell nucleic acid or a portion thereof; and (d) genotyping the fetus by evaluating for a genetic difference compared to maternal cell. The genetic difference may be a copy number variation of a gene or a chromosomal region. The chromosomal region may be less than about 2 Mb in length. The chromosomal region may be less than about 1, about 0.9, about 0.8, about 0.7, about 0.6, about 0.5, about 0.4, about 0.3, about 0.2, or about 0.1 Mb in length. The genetic difference may be a copy number variation of substantially an entire chromosome. The genetic difference may be a translocation. The genetic difference may be a nucleic acid sequence associated with a pathological condition. The nucleic acid sequence associated with the pathological condition may be an allele associated with the pathological condition. The genetic difference may be a polymorphism. The polymorphism may be an indel. The genetic difference may be a single nucleotide polymorphism (SNP). The genetic difference may be mosaicism. The mosaicism may be confined placental mosaicism. The genetic difference may be uniparental disomy. The genetic difference may be twins.

The pathological condition may be lp36 deletion syndrome, 18p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, aceruloplasminemia, acheiropodia, achondrogenesis type II, achondroplasia, acute intermittent porphyria, adenylosuccinate lyase deficiency, adrenoleukodystrophy, alagille syndrome, ADULT syndrome, Aicardi-Goutieres syndrome, Albinism, Alexander disease, alkaptonuria, alport syndrome, alternating hemiplegia of childhood, amyotrophic lateral sclerosis, frontotemporal dementia, alstrom syndrome, amelogenesis imperfecta, aminolevulinic acid dehydratase deficiency porphyria, androgen insensitivity syndrome, angelman syndrome, apert syndrome, arthrogryposis-renal dysfunction-cholestasis syndrome, ataxia telangiectasia, axenfeld syndrome, Beare-Stevenson cutis gyrata syndrome, Beckwith-Wiedemann syndrome, Benjamin syndrome, biotinidase deficiency, Bjornstad syndrome, bloom syndrome, Birt-Hogg-Dube syndrome, brody myopathy, brunner syndrome, CADASIL syndrome, CARASIL syndrome, Chronic granulomatous disorder, campomelic dysplasia, canavan disease, carpenter syndrome, cerebral dysgenesis-neuropathy-ichthyosis-keratoderma syndrome (SEDNIK), cystic fibrosis, charcot-marie-tooth disease, CHARGE syndrome, Chediak-Higashi syndrome, cleidocranial dysostosis, cockayne syndrome, Coffin-Lowry syndrome, Cohen syndrome, collagenopathy, types II and XI, congenital insensitivity to pain with anhidrosis (CIPA), congenital muscular dystrophy, Cornelia de Lange syndrome (CDLS), cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (genetic hypercalciuria), Denys-Drash syndrome, De Grouchy syndrome, down syndrome, Di George's syndrome, Distal hereditary motor neuropathies, multiple types, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Straussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, familial, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type I (Wermer's syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Trisomy 8, Trisomy 9, Trisomy, 22, Tuberous sclerosis complex (TSC), Turner syndrome, Usher syndrome, Variegate porphyria, von Hippel-Lindau disease, Waardenburg syndrome, Weissenbacher-Zweymuller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xpl 1.2 duplication syndrome, X-linked severe combined immunodeficiency (X-SCID), X-linked sideroblastic anemia (XLSA), 47, XXX (triple X syndrome), XXXX syndrome (48, XXXX), XXXXX syndrome (49, XXXXX), XYY syndrome (47,XYY), or Zellweger syndrome.

Amplification of the fetal cell nucleic acid or a portion thereof may comprise whole genome amplification. Genotyping the fetus by evaluating for a genetic difference compared to maternal sample may comprise quantitative polymerase chain reaction amplification (qPCR), SNP array, array comparative genomic hybridization (array CGH), next generation sequencing (NGS), or Short Tandem Repeat analysis (STR analysis). NGS may be a single cell NGS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other objects, features and advantages will be apparent from the following description of particular embodiments of the disclosure, as illustrated in the accompanying drawings. The drawings are not necessarily to scale; emphasis instead being placed upon illustrating the principles of various embodiments of the disclosure.

FIG. 1A-1D are images showing a singlet trophoblast, maternal cells and cell nuclei in various channels used for identification. In FIG. 1A, the blue color shows cell nuclei and the green color shows singlet fetal trophoblast with cytokeratin as the cell marker. FIG. 1B is an image showing overlay of all channels. The blue color shows cell nuclei, the green color shows singlet fetal trophoblast with cytokeratin as the cell marker and the yellow color shows maternal cells with CD45 as the cell marker. FIG. 1C is an image showing cell nuclei. FIG. 1D is an image showing maternal cells. The blue color shows cell nuclei and the yellow color shows maternal cells with CD45 as the cell marker.

FIG. 2A-2D is an image showing a doublet trophoblast, maternal cells and cell nuclei in various channels used for identification. In FIG. 2A, the blue color shows cell nuclei and the green color shows doublet fetal trophoblast with cytokeratin as the cell marker. FIG. 2B is an image showing overlay of all channels. The blue color shows cell nuclei, the green color shows doublet fetal trophoblast with cytokeratin as the cell marker and the yellow color shows maternal cells with CD45 as the cell marker. FIG. 2C is an image showing cell nuclei. FIG. 2D is an image showing maternal cells. The blue color shows cell nuclei and the yellow color shows maternal cells with CD45 as the cell marker.

FIG. 3 is an image showing distinct staining of male cells compared to female cells. In the image, the red color shows the female cell with XIST cell marker and yellow color shows the male cell with TTTY15 cell marker.

FIG. 4 is a graph showing genotyping information. Cells are classified based on comparison of maternal vs. fetal SNP data and established thresholds.

FIG. 5 shows a normal cell, a male cell with trisomy 21 and a female cell with a deletion on chromosome 15 as identified by the methods shown in the disclosure. The fetal cell abnormalities such as trisomies and deletions are identified by the methods shown in the disclosure.

FIG. 6 is a graph showing number of reportable trophoblasts. 91% of the samples can recover trophoblasts by the methods shown in the disclosure.

### DETAILED DESCRIPTION

The primary challenges for developing cell-based NIPT is that the fetal cells are exceedingly rare in maternal blood and is the difficultly in distinguishing between fetal cells from maternal cells. Blood contains about 4-6×10⁹ red blood cells (RBC) per mL, and about 4-6×10⁶/mL white blood cells. In contrast, the expected number of fetal cells in a maternal blood sample is about 1-5 cells/mL. The methods described herein overcome some of the challenges associated with cell-based NIPT.

### I. Fetal Cell Enrichment

In some embodiments, the disclosure provides a method of isolating fetal cells from a biological sample. Such methods may include the steps of (a) contacting the biological sample with one or more binding agents directed against at least one of the group consisting of, a fetal cell marker, a maternal cell marker, and a nuclear marker, (b) sorting the fetal cells of the biological sample based on detection of the one or more binding agents bound to said at least one nuclear marker, a fetal cell marker, or a maternal cell marker, wherein the sorting is performed using a microfluidic cell separator, microbubbles-based cell sorting device, fluorescence activated cell sorting (FACS) device or magnetic activated cell sorting (MACS) device; and (c) determining the presence of the fetal cells as those bound by the binding agents from step (b).

In some embodiments, the biological sample is obtained from a female subject. In some embodiments, the female subject may be pregnant or is suspected to be pregnant. In some embodiments, the female subject may be pregnant with a singleton fetus or with multiple fetuses (twins or higher). The biological sample can be any sample that contains or is likely to contain at least one fetal cell. In some embodiments, the fetal cells can be obtained with no more than negligible risk to the subject or the pregnancy. Suitable biological samples include, without limitation, peripheral blood and cervical secretions.

Biological samples can be obtained during any week of gestation. In some embodiments, the biological sample is obtained from the pregnant woman at a gestational age of less than about 17 weeks. In some embodiments, the gestational age is less than about 17, 16, 15, 14, 13, 12, 11, 10, 9, 8, 7, 6 or 5 weeks. In some embodiments, the gestational age is between about 6 weeks and about 15 weeks. In some embodiments, the gestational age is between about 1 week and about 26 weeks. In some embodiments, the biological sample is obtained between about 3 weeks and about 20 weeks. In some embodiments, the biological sample is obtained between about 5 weeks and about 18 weeks. In some embodiments, the biological sample is obtained before about gestational week 20, 19, 18, 17, 16, 15, 14, 13, 12, 11, 10, 9, 8, 7, 6, 5, or 4. In some embodiments, the biological sample is obtained after about gestational week 1, 2, 3, 4, 5, 6, 7, 8, 9, or 10.

Binding Agents

The term “binding agent” as used herein refers to a polypeptide, nucleic acid or a small or large molecule which interacts with a particular cell or cell-compartment thereof. In some embodiments, the cell may be a maternal or fetal cell. In some embodiments, the binding agent may be any antibody, antibody fragment, a nucleic acid (e.g., probe), aptamer, or a small or large molecule. In some embodiments, the fetal cells are trophoblasts. In some embodiments, the fetal cells may be fetal nucleated red blood cells (fnRBC), fetal lymphocytes (B or T cell), fetal progenitor (stem) cells, or any other fetal cell in the maternal blood. In some embodiments, the cells are maternal cells.

In some embodiments, the fetal cells e.g., trophoblasts may be present in maternal blood. The trophoblasts (placental cells) are believed to enter the bloodstream when the placenta is forming and invading the uterine wall, after implantation of the fetus. In some embodiments, multiple binding agents may be used together.

Nucleotide Probe

In some embodiments, one or more binding agents may be a nucleotide probe. The term “nucleotide probe” as used herein refers to a nucleic acid sequence which is complementary to a nucleic acid sequence that is present or enriched in a fetal cell or a maternal cell.

In some embodiments, the nucleotide probe may be complementary to either the coding strand or the non-coding portion of the gene or complementary to a non-coding region or RNA or DNA. In some embodiments, the nucleotide probe is complementary to the coding strand (non-template strand). In some embodiments, the nucleotide probe is directed to the mRNA. In some embodiments, the nucleotide probe is directed to the fetal mRNA. In some embodiments, the nucleotide probe may be complementary to non-coding RNAs XIST, TTTY15, UTY, KDM5D, DDX3Y, EIF1AY, ZFY, TMSB4Y, USP9Y, RPS4Y1, NLGN4Y. In some embodiments, the nucleotide probe may be complementary to non-coding RNA XIST. In some embodiments, the nucleotide probe may be complementary to non-coding RNA TTTY15.

In some embodiments, the nucleotide probe may be DNA or RNA probe. In some embodiments, the nucleotide probe may be DNA probe. In some embodiments, the nucleotide probe may be RNA probe. In some embodiments, the nucleotide probes may be modified with non-natural nucleotides that improve binding affinity and/or binding specificity. In some embodiments, the non-natural nucleotides may be LNA (locked nucleic acids), TINA (twisted intercalating nucleic acids), PNA (peptide nucleic acid), INA (intercalating nucleic acids), morpholino and 2′O-substituted RNA monomers such as 2′O-methyl RNA monomers and 2′O-(2-methoxyethyl) RNA. Exemplary nucleotide probes include, but are not limited to, ribonucleic acid probes (RNAs), deoxyribonucleic acid probes (DNAs), threose nucleic acid probes (TNAs), glycol nucleic acid probes (GNAs), peptide nucleic acid probes (PNAs), locked nucleic acid probes (LNAs) or hybrids thereof.

In some embodiments, the length of the nucleotide probes may be of any suitable length, such as, but not limited to, in the range of 10 to 200 nucleotides (e.g., from 10 to 20, from 10 to 30, from 10 to 40, from 10 to 50, from 10 to 60, from 10 to 70, 10 to 80, from 10 to 90, from 10 to 100, from 10 to 110, from 10 to 120, from 10 to 130, 10 to 140, from 10 to 150, from 10 to 160, from 10 to 170, from 10 to 180, from 10 to 190, 20 to 200, from 20 to 30, from 20 to 40, from 20 to 50, from 20 to 60, from 20 to 70, 20 to 80, from 20 to 90, from 20 to 100, from 20 to 110, from 20 to 120, 20 to 130, from 20 to 140, from 20 to 150, from 20 to 160, from 20 to 170, 20 to 180, from 20 to 190, from 30 to 200, from 30 to 40, from 30 to 50, 30 to 70, from 30 to 100, 30 to 150, and from 30 to 190 nucleotides).

In some embodiments, the nucleotide probe is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% complementary to the fetal mRNA. In some embodiments, the nucleotide probe is at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary to the fetal mRNA.

In some embodiments, the nucleotide probe may be immobilized on a support. In some embodiments, the nucleotide probe-immobilized support may be used in a known apparatus such as RNA/DNA chip and RNA/DNA microarray.

In some embodiments, the nucleotide probe sequences may be used to analyze fetal cells using in situ hybridization (ISH) assay as known to one skilled in the art. In situ hybridization assays are well known and are generally described in Angerer et al., Methods Enzymol. 152:649-660 (1987). In an in situ hybridization assay, cells are fixed to a solid support, typically a glass slide. The cells are then contacted with a hybridization solution at a moderate temperature to permit annealing of specific probes that may be labeled. The probes are generally labeled with radioisotopes or fluorescent reporters.

In some embodiments, the nucleotide probe sequences may be used to analyze fetal cells using fluorescent in situ hybridization (FISH) assay as known to one skilled in the art. FISH uses fluorescent probes that bind to only those parts of a sequence with which they show a high degree of sequence similarity. FISH is a cytogenetic technique used to detect and localize specific polynucleotide sequences in cells. For example, FISH can be used to detect DNA sequences on chromosomes or specific RNAs, e.g., mRNAs, within tissue samples. Fluorescence microscopy can be used to find out whether and where the fluorescent probes are bound.

In some embodiments, the nucleotide probe sequences may be used to analyze fetal cells using RNAscope™, DNAcope™, and BaseScope™ as specialized highly sensitive variations on ISH. Generally, these methods amplify the signal greatly and can give much improved sensitivity and specificity.

In some embodiments, the nucleotide probe may include at least two target probes and a probe system. In some embodiments, the at least two target probes are capable of hybridizing to non-overlapping sequences in fetal mRNA. In some embodiments, the at least two target probes are also capable of hybridizing to the probe system. In some embodiments, each of the at least two target probes comprises a first polynucleotide sequence F-1 that is complementary to a polynucleotide sequence in fetal mRNA and a second polynucleotide sequence F-2 that is complementary to a polynucleotide sequence in the probe system.

Generally, the target probe as used herein refers to a polynucleotide that is capable of hybridizing to a nucleic acid of interest and to a probe system. The target probe typically has a first polynucleotide sequence F-1, which is complementary to a polynucleotide sequence of the nucleic acid of interest, and a second polynucleotide sequence F-2, which is complementary to a polynucleotide sequence of the probe system.

The probe system as used herein comprises one or more polynucleotides that collectively comprise a label and at least two polynucleotide sequences M-1, each of which is capable of hybridizing to a target probe. The label provides a signal, directly or indirectly. Polynucleotide sequence M-1 is typically complementary to sequence F-2 in the target probe. In some embodiments, the probe system can include a plurality of label probes, amplification multimer and a preamplifier.

The amplification multimer as used herein is a polynucleotide comprising a plurality of polynucleotide sequences M-2. Polynucleotide sequence M-2 is complementary to a polynucleotide sequence in the label probe. The amplification multimer may also include at least one polynucleotide sequence that is capable of hybridizing to a target probe or to a nucleic acid that hybridizes to the target probe, e.g., a preamplifier.

The label probe as used herein is a single-stranded polynucleotide that comprises a label that directly or indirectly provides a detectable signal. The label probe generally comprises a polynucleotide sequence that is complementary to the repeating polynucleotide sequence M-2 of the amplification multimer. However, if no amplification multimer is used, the label probe can hybridize directly to a target probe.

The preamplifier as used herein is a polynucleotide that serves as an intermediate between one or more target probes and amplification multimers. Generally, the preamplifier is capable of hybridizing simultaneously to at least two target probes and to a plurality of amplification multimers.

In some embodiments, the nucleotide probe sequences, or the nucleotide probe components may be any sequences described in the US patent publications or patent numbers U.S. Pat. Nos. 7,709,198, 8,604,182, or US20160201117, the contents of each of which are incorporated by reference in their entirety. In some embodiments, the nucleotide probes or the nucleotide probe components may be any probes or components used in RNAscope™ developed by Advanced Cell Diagnostics Inc, https://acdbio.com/products. In some embodiments, the nucleotide probes or the nucleotide probe components may be any nucleotide probes used in PRIMEFLOW™ developed by ThermoFisher Scientific. In some embodiments, the nucleotide probe sequences, or the nucleotide probe components may be any sequences described in M. B. Hanley et al., PLOS One (2013) 8(2):e57002, the contents of which are incorporated by reference in its entirety.

Antibody

In some embodiments, the one or more binding agents may be an antibody or a fragment thereof. As used herein, the term “antibody” is referred to in the broadest sense and specifically covers various embodiments, including, but not limited to, polyclonal antibodies, monoclonal antibodies, multispecific antibodies (e.g., bispecific antibodies or trispecific antibodies), human antibodies, humanized antibodies, chimeric antibodies, single chain antibodies, single chain Fv (scFv) formats, diabodies, intrabodies, unibodies, maxibodies, and antibody fragments. As used herein the term “antibody fragment” refers to a portion of a whole antibody or a fusion protein that includes such a portion. Antibody fragments may include antigen binding regions. In some embodiments, antibody fragments include, but are not limited to Fab fragments, Fab′ fragments, F(ab′)₂ fragments, Fv fragments, Fc fragments, variable domains, constant domains, heavy chains, and light chains. In some embodiments, antibody fragments may be prepared by enzymatic digestion. Fab fragments may be prepared by papain digestion of whole antibodies. F(ab′)₂ fragments may be prepared by pepsin treatment of whole antibodies. Antibodies are primarily amino-acid based molecules but may also include one or more modifications (including, but not limited to sugar moieties, fluorescent labels, chemical tags, etc.).

In some embodiments, the antibody or a fragment thereof may bind to a fetal cell. In some embodiments, the antibody or a fragment thereof may bind to a trophoblast. In some embodiments, the antibody or a fragment thereof may bind to a protein expressed on the surface of any fetal cell. In some embodiments, the antibody or a fragment thereof may bind to a protein expressed in the cytoplasm of any fetal cell. In some embodiments, the antibody or a fragment thereof may bind to a protein expressed on in the nucleus of any fetal cell. In some embodiments, the fetal cells may be fetal nucleated red blood cells (fnRBC), fetal lymphocytes (B or T cell), fetal progenitor (stem) cells, or any other fetal cell in the maternal blood.

In some embodiments, the antibody or a fragment thereof may bind to a protein expressed on the surface of a fetal epithelial cell. In some embodiments, the antibody or a fragment thereof may bind to a protein expressed in the cytoplasm of a fetal epithelial cell. In some embodiments, the antibody or a fragment thereof may bind to a protein expressed in the nucleus of a fetal epithelial cell. In some embodiments, the antibody or a fragment thereof may bind to trophoblast specific antigens. In some embodiments, the antibody or a fragment thereof may bind to a cytokeratin, human chorionic gonadotropin (HCG), human placental lactogen (HPL) or SP₁. In some embodiments, the antibody or a fragment thereof may bind to T-cells, B-cells, or hematopoietic stem and progenitor cell (HSPC).

In some embodiments, the antibody or a fragment thereof may bind to a cytokeratin. In some embodiments, the antibody or a fragment thereof may bind to Human Cytokeratin 1 (CK1), Human Cytokeratin 2 (CK2), Human Cytokeratin 3 (CK3), Human Cytokeratin 4 (CK4), Human Cytokeratin 5 (CK5), Human Cytokeratin 6 (CK6), Human Cytokeratin 7 (CK7), Human Cytokeratin 8 (CK8), Human Cytokeratin 9 (CK9), Human Cytokeratin 10 (CK10), Human Cytokeratin 13 (CK13), Human Cytokeratin 14 (CK14), Human Cytokeratin 15 (CK15), Human Cytokeratin 16 (CK16), Human Cytokeratin 17 (CK17), Human Cytokeratin 18 (CK18), or Human Cytokeratin 19 (CK19).

In some embodiments, the antibody or a fragment thereof may bind to a protein expressed on the surface of a fetal endothelial cell. In some embodiments, the antibody or a fragment thereof may bind to a protein expressed in the cytoplasm of a fetal endothelial cell. In some embodiments, the antibody or a fragment thereof may bind to a protein expressed in the nucleus of a fetal endothelial cell. In some embodiments, the antibody or a fragment thereof may bind to CD-105, CD146 or CD141. In some embodiments, the antibody or a fragment thereof may bind to CD-105.

In some embodiments, the antibody or a fragment thereof may bind to RPS4Y1. The RPS4Y1 protein is generally found in transcriptionally active ribosomes extracted from placenta of a male fetus and expressed in testis and in several somatic tissues of male individuals. The sequence of RPS4Y1 protein, SEQ ID NO: 1 is shown below.

MARGPKKHLKRVAAPKHWMLDKLTGVFAPRPSTGPHKLRECLPLIVFLRNRL KYALTGDEVKKICMQRFIKIDGKVRVDVTYPAGFMDVISIEKTGEHFRLVYDTKGRFAV HRITVEEAKYKLCKVRKITVGVKGIPHLVTHDARTIRYPDPVIKVNDTVQIDLGTGKIINF IKFDTGNLCMVIGGANLGRVGVITNRERHPGSFDVVHVKDANGNSFATRLSNIFVIGNG NKPWISLPRGKGIRLTVAEERDKRLATKQSSG (SEQ ID NO: 1). In some embodiments, the antibody or a fragment thereof may bind to amino acids 155-177 of SEQ ID NO: 1. In some embodiments, the antibody or a fragment thereof may include formats according to any of those described in Spena, S. et al. 2021 Int. J. Mol. Sci. 22: doi 10.3390/ijms22042001, the contents of which are herein incorporated by reference in their entirety. In some embodiments, the antibody or a fragment thereof may be obtained from commercial sources. In some embodiments, the antibody or a fragment thereof which can bind to RPS4Y1 may be obtained from commercial sources. In some embodiments, the antibody or a fragment thereof which can bind to RPS4Y1 may be PAS-38976 obtained from ThermoFisher Scientific. In some embodiments, the antibody or a fragment thereof which can bind to RPS4Y1 may be ab74709 obtained from Abcam. In some embodiments, the antibody or a fragment thereof which can bind to RPS4Y1 may be DCABH-13326 obtained from Creative Diagnostics. In some embodiments, the antibody or a fragment thereof which can bind to RPS4Y1 may be commercially obtained from United States Biological, Mybiosource, Abcam, ThermoFisher Scientific or Creative Diagnostics.

In some embodiments, the present disclosure includes antibody variants. As used herein, the term “antibody variant” refers to an antibody that includes at least one modification relative to a native antibody or starting antibody. Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments.

In some embodiments, the antibody variant may be an antibody that includes at least one modification relative to the antibodies described in the journal publication Spena, S. et al. In some embodiments, the antibodies binding to RPS4Y1 protein described in the present disclosure may be optimized to provide a higher binding affinity for RPS4Y1 relative to the antibodies described in the journal publication Spena, S. et al.

The variable domains are specific antibody domains found on both the antibody heavy and light chains that differ extensively in sequence among antibodies and determine antibody specificity for particular antigens. Variable domains include hypervariable regions that include amino acid residues responsible for antigen binding. Amino acids present within hypervariable regions determine the structure of complementarity determining regions (CDRs) that become part of the antigen-binding site of the antibody. As used herein, the term “CDR” refers to an antibody region having a structure that is complimentary to its target antigen or epitope. In some embodiments, the CDR of an antibody is optimized to provide a higher binding affinity for RPS4Y1 relative to the antibodies described in the journal publication Spena, S. et al.

In some embodiments, the fetal cell is a male fetal cell. In some embodiments, the male fetal cells are sorted by any of the methods used herein. In some embodiments, the male fetal cells are sorted by any of the binding agents used herein. In some embodiments, the fetal cell is a female fetal cell. In some embodiments, the female fetal cells are sorted by any of the methods used herein. In some embodiments, the female fetal cells are sorted by any of the binding agents used herein.

In some embodiments, the antibody or a fragment thereof may bind to human leucocyte antigen (HLA) or other polymorphic markers where the fetal cell has an allele inherited from the father which allele is not present in the mother, L. A. Herzenberg, 1979 Proc. Natl. Acad. Sci. 76:1453-1455, the contents of which are herein incorporated by reference in its entirety.

In some embodiments, the antibody or a fragment thereof may bind to a maternal cell. In some embodiments, the antibody or a fragment thereof may bind to a trophoblast and a maternal cell. In some embodiments, the antibody or a fragment thereof may bind to a protein expressed on the surface of a maternal cell. In some embodiments, the antibody or a fragment thereof may bind to a protein expressed in the cytoplasm of a maternal cell. In some embodiments, the antibody or a fragment thereof may bind to a protein expressed in the nucleus of a maternal cell. In some embodiments, the antibody or a fragment thereof may bind to CD45.

In some embodiments, the antibody or a fragment thereof may have a high binding affinity for the selected cell marker. In some embodiments, the antibody of the present disclosure binds to the protein when the dissociation constant K_{d} is ≤10⁻⁸M. The term dissociation constant (K_{D}) as used herein refers to an equilibrium constant that measures the propensity of a complex of associated molecules to separate (dissociate) reversibly into the separate molecules. In some embodiments, the antibody or a fragment thereof may have Kd of less than 10⁻²M, less than 10⁻³M, less than 10⁻⁴M, less than 10⁻⁵M, less than 10⁻⁶M, less than 10⁻⁷M, less than 10⁻⁸M, less than 10⁻⁹M, less than 10⁻¹⁰M, less than 10⁻¹¹M, less than 10⁻¹²M, less than 10⁻¹³M, less than 10⁻¹⁴M, or less than 10⁻¹⁵M.

In some embodiments, antibodies of the present disclosure include multispecific antibodies. As used herein, the term “multispecific antibody” or “multibody” refers to an antibody capable of binding two or more targets. The targets may be distinct structures or may be distinct regions or epitopes of a single structure. Multispecific antibodies may include bispecific antibodies. As used herein, the term “bispecific antibody” refers to an antibody capable of binding two different targets or antigens. Such antibodies typically include antigen binding regions from at least two different antibodies. Bispecific antibodies may include formats according to any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

Antibodies of the present disclosure may include diabodies. As used herein, the term “diabody” refers to a small antibody fragment with two antigen-binding sites. Diabodies include a heavy chain variable domain VH connected to a light chain variable domain VL in the same polypeptide chain. By using a linker that is too short to allow pairing between the two domains on the same chain, the domains are forced to pair with the complementary domains of another chain and create two antigen-binding sites. Diabodies are described more fully in, for example, EP404097; WO1993/011161; and Hollinger et al. (Hollinger, P. et al., “Diabodies”:Small bivalent and bispecific antibody fragments. PNAS. 1993. 90:6444-8) the contents of each of which are incorporated herein by reference in their entirety.

In some embodiments, antibodies of the present disclosure include monoclonal antibodies. As used herein, the term “monoclonal antibody” refers to an antibody obtained from a population of substantially homogeneous cells (or clones), i.e., the individual antibodies included in the population are identical and/or bind the same epitope. In contrast to polyclonal antibody preparations that typically include different antibodies directed against different determinants (epitopes), each monoclonal antibody is directed against a single epitope.

The modifier “monoclonal” indicates the character of the antibody as being obtained from a substantially homogeneous population of antibodies, and is not to be construed as requiring production of the antibody by any particular method. Monoclonal antibodies may include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

Antibodies of the present disclosure may include or be formatted as humanized antibodies. As used herein, the term “humanized antibody” refers to a chimeric antibody including a minimal portion from one or more non-human (e.g., murine) antibody source with the remainder derived from one or more human immunoglobulin source. For the most part, humanized antibodies are human immunoglobulins in which hypervariable region residues are replaced by hypervariable region residues from non-human “donor” antibodies, e.g., from a mouse, rat, rabbit or nonhuman primate antibody having a desired specificity, affinity, capacity, and/or other binding characteristic.

In some embodiments, antibodies of the present disclosure include antibody mimetics. As used herein, the term “antibody mimetic” refers to any molecule which mimics the function or effect of an antibody, and which binds specifically and with high affinity to their molecular targets. In some embodiments, antibody mimetics include monobodies, designed to incorporate the fibronectin type III domain (Fn3) as a protein scaffold (e.g., see U.S. Pat. Nos. 6,673,901 and 6,348,584). Antibody mimetics may include, but are not limited to affibody molecules, affilins, affitins, anticalins, avimers, Centyrins, DARPINS™, Fynomers and Kunitz and domain peptides. In other embodiments, antibody mimetics may include one or more non-peptide region.

In some embodiments, antibodies of the present disclosure may include “unibodies,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. Other antibodies may be “miniaturized” antibodies, which are compacted 100 kDa antibodies (see, e.g., Nelson, A. L., MAbs., 2010. January-Febuary; 2(1):77-83).

Various antibody preparation methods are known in the art. Techniques for the antibody production include those described, e.g., in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

Microfluidic Cell Separator

In some embodiments, the disclosure provides a method for sorting cells. Generally, the term sorting or enriching refers to detection or isolation of one or more cell or cell types(s) in a population of cells. In some embodiments, the device may use a microfluidic technology. In some embodiments, the disclosure provides a method for sorting fetal cells using a device. In some embodiments, the device may use a combination of microfluidics, flow cytometry and liquid dispensing technology.

In some embodiments, the fetal cells are sorted by a microfluidic cell separator. In some embodiments, the microfluidic cell separator sorts the fetal cells at a pressure less than about 20 psi. In some embodiments, the pressure is less than about 20 psi, 15 psi, lOpsi, 9 psi, 8 psi, 7 psi, 6 psi, 5 psi, 4 psi, 3 psi, 2 psi, 1.5 psi, 1 psi, 0.5 psi, 0.4 psi, 0.3 psi, 0.2 psi, or 0.1 psi. In some embodiments, the microfluidic cell separator sorts the fetal cells at a pressure less than about 2 psi.

In some embodiments, the microfluidic cell separator comprises a sheath fluid. The term sheath fluid as used herein refers to a carrier fluid that runs in a microfluidic cell separator. In some embodiments, a suspension of fetal cells can be injected into the center of a flowing sheath fluid. In some embodiments, sheath fluid has a flow rate of from about 5 mL/h to about 10 mL/h. The flow rate as defined herein refers to the volume of the fluid which passes per unit time. In some embodiments, the flow rate is of from about 5 mL/h, 5.5 mL/h, 6 mL/h, 6.5 mL/h, 7 mL/h, 7.5 mL/h, 8 mL/h, 8.5 mL/h, 9 mL/h, 9.5 mL/h to about 10 mL/h. In some embodiments, the flow rate is about 6.5 mL/h.

In some embodiments, the microfluidic cell separator comprises a droplet size of from about 0.5 μL to about 2.0 μL. In some embodiments, the droplet size is from about 0.5 μL, 0.6 μL, 0.7 μL, 0.8 μL, 0.9 μL, 1.0 μL, 1.1 μL, 1.2 μL, 1.3 μL, 1.4 μL, 1.5 μL, 1.6 μL, 1.7 μL, 1.8 μL, 1.9 μL to about 2 μL. In some embodiments, the droplet size is about 1.0 μL.

In some embodiments, the microfluidic cell separator has a processing speed of from about 2 to about 50,000 cells/second. In some embodiments, the processing speed is from about 2, 20, 50, 100, 150, 200, 300, 400, 500, 600, 700, 800, 900, 1,000, 2,000, 3,000, 4,000, 5,000, 6,000, 7,000, 8,000, 9,000, 10,000, 15,000, 20,000, 25,000, 30,000, 35,000, 40,000, 45,000 to about 50,000 cells/second.

In some embodiments, the fetal cells may be sorted using a device described in the U.S. Patent Nos. 9,702,808 or 8,820,532, the contents of each of which are incorporated by reference in their entirety. In some embodiments, the fetal cells may be sorted using a Namocell Namo™, Hana™, or Pala™ (Namocell Inc., Mountain View, CA) devices. Generally, high-density samples containing the target cells are first suspended and loaded into Namocell's microfluidic cell cartridge. Using the enrichment sorting mode, the cells of interest are dispensed and collected into a single well, producing a pool of cells enriched for the target population. Subsequently, these enriched cells are loaded into a new cell cartridge and dispensed using the single cell sorting mode to be collected as singlets in a 96-well plate.

In some embodiments, the fetal cells may be sorted by a fluorescence activated cell sorting (FACS) device. FACS can be used to sort cells into two or more containers. FACS can be based on the light scattering and fluorescent characteristics of each type of cell. A suspension of particles (e.g., cells) can be entrained in a flowing stream of liquid. There can be separation between particles in the liquid. The stream of particles (e.g., cells) can be broken into droplets (e.g., by a vibrating mechanism). In some embodiments, only one particle (e.g., cell) is in each droplet. In some embodiments, before the stream breaks into droplets, the liquid passes through a fluorescence measuring station. The fluorescence characteristics can be measured. A charge can be given to each droplet based on the fluorescence measurement, and the charged droplets can pass through an electrostatic deflection system that can divert droplets to containers based on charge. In some embodiments, the fetal cells may not be sorted by a fluorescence activated cell sorting (FACS) device.

Microbubble Technology

In some embodiments, the fetal cells are sorted using a microbubbles-based cell sorting device. The device may enable sorting of fetal cells by microbubble technology. The device may comprise a system having a binding agent that specifically binds a fetal cell protein, where the binding agent is linked to a microbubble, and the microbubble has sufficient buoyancy to enable efficient separation of the fetal cells from the bulk cells such as maternal cells in a biological sample. In some embodiments, the fetal cells are sorted by any of the methods shown in the International Patent Publication WO 2021/154965, the contents of which are incorporated in reference to its entirety.

In some embodiments, microbubbles of various sizes and buoyancy are used. In some embodiments, microbubbles are composed of a rigid material, for example without limitation, glass, polystyrene, and the like. In an embodiment, the microbubbles are composed of glass. In another embodiment, the microbubbles comprise polymers. In an embodiment, the microbubbles comprise polystyrene. In another embodiment, the microbubbles comprise a mixture of glass microbubbles and polymer microbubbles.

In some embodiments, the microbubbles may be solid. In some embodiments, the microbubbles may be hollow. Hollow microbubbles may have a wall thickness sufficient to make the microbubble rigid and capable of resisting breakage or rupture when handled under reasonable laboratory conditions, for example under pressures of from about 0.2 to about 5 atm. In an embodiment, hollow microbubbles have an average wall thickness of from about 0.2 μm to 2.0 μm. In an embodiment, hollow microbubbles have an average wall thickness of from about 0.4 μm to 1.0 μm. In an embodiment, hollow microbubbles have an average wall thickness of from about 0.5 μm to 0.8 μm. In an embodiment, the microbubbles have an average wall thickness of about 0.7 μm. Hollow microbubbles can also comprise a gas, liquid, or vacuum interior. Non-limiting examples of the gas include hydrogen, helium, nitrogen, oxygen, neon and CO₂. In some embodiments, suitable gases can be selected that do not degrade the microbubble walls.

In some embodiments, the microbubbles may have a density less than the density of the samples from which fetal cells will be enriched in order to be buoyant. Generally, the microbubbles have a density low enough such that when bound to a fetal cell, the combination of microbubble and fetal cell together (including the binding agent) is more buoyant than the non-fetal cells in the biological sample, to a degree sufficient to separate it from the non-fetal cells. In some embodiments, the buoyancy of the microbubbles enable them to be suspended in the bulk biological sample long enough to interact with the fetal cells and/or binding agents. In some embodiments, the buoyancy is such that the microbubbles will not separate bound fetal cells from the bulk of the biological sample slowly, or fail to effect a complete separation. In an embodiment, the microbubbles have a density that is at least about 20%, 25%, 30%, 35%, 40%, 45%, 50%, 55%, 60%, 65%, 70%, 75%, or 80% of the density of the biological sample. In an embodiment, the microbubbles have a density that is no more than about 98%, 95%, 90%, 85%, 80%, 75%, 70%, 65%, 60%, 55%, or about 50% of the density of the biological sample. In an embodiment, the microbubbles have a density that is between about 40% and about 80% of the density of the biological sample. In an embodiment, the microbubbles have a density that is between about 50% and about 75% of the density of the biological sample. In an embodiment, the microbubbles have a density that is about 70% of the density of the biological sample. In an embodiment, the microbubbles have a density that is between about 0.20 g/cm3 and 0.90 g/cm3. In an embodiment, the density is about 0.20, 0.30, 0.40, 0.50, 0.60, 0.65, 0.70, 0.75, 0.80, 0.85, or 0.90 g/cm³. In an embodiment, the microbubbles have a density that is at least about 20%, 25%, 30%, 35%, 40%, 45%, 50%, 55%, 60%, 65%, 70%, 75%, or 80% of the density of the non-fetal cells in the biological sample. In an embodiment, the microbubbles have a density that is no more than about 98%, 95%, 90%, 85%, 80%, 75%, 70%, 65%, 60%, 55%, or about 50% of the density of the non-fetal cells in the biological sample. In an embodiment, the microbubbles have a density that is at least about 20%, 25%, 30%, 35%, 40%, 45%, 50%, 55%, 60%, 65%, 70%, 75%, or 80% of the density of sample medium (i.e., the fluid component of the biological sample, excluding fetal and non-fetal cells). In an embodiment, the microbubbles have a density that is no more than about 98%, 95%, 90%, 85%, 80%, 75%, 70%, 65%, 60%, 55%, or about 50% of the density of the sample medium.

Microbubbles can be prepared using methods known in the art (see, for example, US patent publication US 2016/0152513, the contents of which are incorporated in reference in its entirety), or can be obtained from commercial sources (for example, 3M, Akadeum Life Sciences).

Binding agents can be bound to microbubbles directly or indirectly. For example, the binding agent can be coupled chemically on a suitable functional group present on the surface of the microbubble. One can employ methods described in the art to attach covalent linking groups to the binding agent and a microbubble (see, e.g., K. Tsuchikama et al., Protein Cell (2018) 9(1):33-46; Jablonski et al., US 2011/0236884). Alternatively, the binding agent can comprise a first linking agent which binds specifically to a second linking agent that is bound to the microbubble. The first and second linking agents can form a covalent or non-covalent bond. The first linking agent can be, for example without limitation, an epitope that is recognized by the second linking agent, or a compound such as but not limited to biotin, avidin, or streptavidin. The second linking agent can also be, for example without limitation, an epitope that is recognized by the first linking agent, or a compound such as but not limited to biotin, avidin, or streptavidin, wherein the first and second linking agents are selected for binding to each other. In some embodiments, microbubbles are coated with streptavidin. In some embodiments, microbubbles are detachable.

In some embodiments, about one to about 5 different binding agents can be selected. The binding agents can be antibodies which are selected for binding to fetal cell markers (for example, the trophoblast, fetal nucleated red blood cells (fnRBC), fetal lymphocytes (B or T cell), fetal progenitor (stem) cells, or any other fetal cell in the maternal blood), targeting proteins such as, for example, HLA-E, HLA-G, MCAM (CD146), ATG9B, EpCAM, TROP-2, CD31, CD141, CD144, MMP9, ITGAI, CSHI, CD105, LVRN, EGFR, ErbB2, ErbB3, ErbB4, or annexin A4.

The fetal cell-binding agent-microbubble complexes can be separated from other cells and components of the biological sample using the buoyancy of the microbubble. This can be accomplished by mixing the microbubbles with the biological sample which can be accelerated by centrifugation. The fetal cell-binding agent-microbubble complexes float to the top of the biological sample, where they can be separated from the biological sample by aspiration or other techniques to provide a cell population that is enriched in fetal cells.

If desired, the cells can be dissociated from the microbubbles after the separation. The dissociation method employed will generally depend on the nature of the binding agents employed. In some embodiments, where the binding agent is an antibody, it can be removed by changing the pH, or competition with a protein or peptide that is bound by the antibody. Alternatively, antibodies can be cleaved with known enzymes, such as for example, pepsin, papain, FabRICATOR®, FabALACTICA® (Genovis Inc., Cambridge, MA), and similar enzymes in order to release the cell from the microbubble complex. In some embodiments, where the linker is an oligonucleotide, a nuclease can be used to dissociate the cells from the microbubbles.

FACS and MACS

In some embodiments, the fetal cells are enriched by magnetic activated cell sorting (MACS), wherein the binding agents are immobilized on magnetic beads. The cells bound to the binding agents can be separated from non-binders by selecting the particles using magnetism.

In some embodiments, the disclosure provides a step for further enriching a cell population containing circulating fetal cells. In some embodiments, the fetal cells can be further enriched by a microfluidic cell separator, microbubbles-based cell sorting device, fluorescence activated cell sorting (FACS) device or magnetic activated cell sorting (MACS) device.

In some embodiments, the disclosure provides a method further comprising a step of isolating the fetal cells using a single-cell picking device. In some embodiments, the cells are mechanically picked using desired instruments such as, for example, a CytePicker™ retrieval module (RareCyte, Inc., Seattle, WA).

Cell Markers

In some embodiments, the one or more binding agents is directed against a cell marker. In some embodiments, the one or more binding agents is directed against a fetal cell marker. The term fetal cell marker as used herein refers to a nucleic acid, a protein, an oligosaccharide or a glycoprotein enriched in the fetal cells compared to a reference cell e.g., a maternal cell.

In some embodiments, the fetal cell marker is a fetal cell nucleic acid marker. The term fetal cell nucleic acid marker as used herein refers to a nucleic acid enriched in a fetal cell compared to a reference cell e.g., a maternal cell. In some embodiments, the fetal cell specific RNA sequence may be used as the fetal cell marker. The detection of these RNAs, can identify cells of fetal origin. The binding agents used to attach fetal cells in a biological sample containing fetal and maternal cells include nucleic acid molecules, which comprise the nucleotide sequence complementary to the fetal mRNA encoding a specific protein.

In some embodiments, the fetal cell nucleic acid marker is XIST (ENSMBL ID: ENSG00000229807.13), TTTY15 (ENSMBL ID: ENSG00000114374), RPS4Y1 (ENSMBL ID: ENSG00000129824.16), UTY, KDM5D, DDX3Y, EIF1AY, ZFY, TMSB4Y, USP9Y, RPS4Y1, NLGN4Y, KRT7 (ENSMBL ID: ENSG00000135480.17), EPCAM (ENSMBL ID: ENSG00000119888.11), HLA-G (ENSMBL ID: ENSG00000204632.13), ENG (ENSMBL ID: ENSG00000106991.14), or βHCG.

In some embodiments, the fetal cell nucleic acid marker is XIST.

In some embodiments, the fetal cell nucleic acid marker is TTTY15.

In some embodiments, the fetal cell marker is RPS4Y1 protein. The RPS4Y1 protein is generally found in transcriptionally active ribosomes extracted from placenta of a male fetus and expressed in testis and in several somatic tissues of male individuals. In some embodiments, the binding agent may be an antibody or a fragment thereof that binds to the RPS4Y1 protein. The sequence of RPS4Y1 protein, SEQ ID NO: 1 is shown below.

MARGPKKHLKRVAAPKHWMLDKLTGVFAPRPSTGPHKLRECLPLIVFLRNRL KYALTGDEVKKICMQRFIKIDGKVRVDVTYPAGFMDVISIEKTGEHFRLVYDTKGRFAV HRITVEEAKYKLCKVRKITVGVKGIPHLVTHDARTIRYPDPVIKVNDTVQIDLGTGKIINF IKFDTGNLCMVIGGANLGRVGVITNRERHPGSFDVVHVKDANGNSFATRLSNIFVIGNG NKPWISLPRGKGIRLTVAEERDKRLATKQSSG (SEQ ID NO: 1). In some embodiments, the antibody or a fragment thereof may bind to amino acids 155 -177 of SEQ ID NO: 1. In some embodiments, the antibody or a fragment thereof may include formats according to any of those described in Spena, S. et al. 2021 Int. J. Mol. Sci. 22: doi 10.3390/ijms22042001, the contents of which are herein incorporated by reference in their entirety. In some embodiments, the antibody or a fragment thereof may be obtained from commercial sources. In some embodiments, the antibody or a fragment thereof which can bind to RPS4Y1 may be obtained from commercial sources. In some embodiments, the antibody or a fragment thereof which can bind to RPS4Y1 may be PA5-38976 obtained from ThermoFisher Scientific. In some embodiments, the antibody or a fragment thereof which can bind to RPS4Y1 may be ab74709 obtained from Abcam. In some embodiments, the antibody or a fragment thereof which can bind to RPS4Y1 may be DCABH-13326 obtained from Creative Diagnostics. In some embodiments, the antibody or a fragment thereof which can bind to RPS4Y1 may be commercially obtained from United States Biological, Mybiosource, Abcam, ThermoFisher Scientific or Creative Diagnostics.

In some embodiments, the present disclosure includes antibody variants. As used herein, the term “antibody variant” refers to an antibody that includes at least one modification relative to a native antibody or starting antibody. Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments. In some embodiments, the antibody variant may be an antibody that includes at least one modification relative to the antibodies described in the journal publication Spena, S. et al. In some embodiments, the antibodies binding to RPS4Y1 protein described in the present disclosure may be optimized to provide a higher binding affinity for RPS4Y1 relative to the antibodies described in the journal publication Spena, S. et al.

The variable domains are specific antibody domains found on both the antibody heavy and light chains that differ extensively in sequence among antibodies and determine antibody specificity for particular antigens. Variable domains include hypervariable regions that include amino acid residues responsible for antigen binding. Amino acids present within hypervariable regions determine the structure of complementarity determining regions (CDRs) that become part of the antigen-binding site of the antibody. As used herein, the term “CDR” refers to an antibody region having a structure that is complimentary to its target antigen or epitope. In some embodiments, the CDR of an antibody is optimized to provide a higher binding affinity for RPS4Y1 relative to the antibodies described in the journal publication Spena, S. et al.

In some embodiments, the fetal cell is a male fetal cell. In some embodiments, the male fetal cells are sorted by any of the methods used herein. In some embodiments, the male fetal cells are sorted by any of the binding agents used herein. In some embodiments, the binding agent may be an antibody or a fragment thereof that binds to the RPS4Y1 protein.

In some embodiments, the fetal cell marker is a fetal cell surface marker. The term fetal cell surface marker as used herein refers to a protein enriched on the surface of the fetal cells compared to a reference cell e.g., a maternal cell. Non-limiting examples of fetal cell surface markers include CD105, EPCAM, HLA-G TROP2. In some embodiments, the fetal cell marker is a fetal cell cytoplasmic marker. The term fetal cell cytoplasmic marker as used herein refers to a protein enriched in the cytoplasm of the fetal cells compared to the nucleus of a fetal cell or a maternal cell. Non-limiting example of cytoplasmic markers include cytokeratins. In some embodiments, the fetal cell marker is a fetal cell nuclear marker. The term fetal cell nuclear marker as used herein refers to a protein enriched in the nucleus of the fetal cells compared to the cytoplasm of a fetal cell or a maternal cell. Non-limiting example of nuclear marker includes XIST. Generally, fetal cell cytoplasmic markers and fetal cell nuclear markers require that the cells be permeabilized to allow access to the binding agent.

In some embodiments, the fetal cell marker is a fetal epithelial cell marker or a fetal endothelial cell marker. In some embodiments, the fetal cell marker is a fetal epithelial cell marker. Epithelial cell as used herein refers to a cell that line hollow organs as well as those that make up glands and the outer surface of the body. The term fetal epithelial cell marker as used herein refers to a protein enriched in the fetal epithelial cells compared to other fetal cells or reference cells. Endothelial cells comprise the lining of the blood vessels and are important for a variety of processes in the body. For example, endothelial cells play roles in angiogenesis, regulation of blood pressure, blood clotting, inflammation, and filtration. Endothelial cells are a heterogeneous group of cells and may have a variety of characteristics depending upon vessel size, specification to a specific organ, and morphology. The term fetal endothelial cell marker as used herein refers to a protein enriched in the fetal endothelial cells compared to other fetal cells or reference cells. In some embodiments, the binding agent is specific to a cell surface marker. In some embodiments, the binding agent is specific to an intracellular cytoplasmic or nuclear marker. In some embodiments, the fetal cell marker is a fetal epithelial cell marker and wherein the fetal epithelial cell marker is cytokeratin, ALCAM/CD166 (ENSMBL ID: ENSG00000170017.12), Aminopeptidase N/ANPEP, CD13 (ENSMBL ID: ENSG00000166825.15), Alanyl Aminopeptidase, Basal Bodies of Cilia (LhS28), Basal Cell Cytokeratin, beta-Crystallin, beta-Defensin 2, beta-Defensin 3, Bmi-1 oncoprotein, BRCA1 (ENSMBL ID: ENSG00000012048.23), BTEB1, Calcitonin Gene-Related Peptide (CGRP), Calcyclin, Carcinoembryonic Antigen (CEA), Cathepsin E (CaE), Caveolin-1, CD138 (Syndecan-1) (ENSMBL ID: ENSG00000115884.11), CD151 (ENSMBL ID: ENSG00000177697.19), CD46 (ENSMBL ID: ENSG00000117335.20), Clara cell-specific protein, Connexin-43 (Cx43), Cornulin, CRNN (ENSMBL ID: ENSG00000143536.7), C1ORF10, SEP53, Cystatin C, Desmin, Desmocollin 2, Desmocollin 3, E-Cadherin, Epithelial Antigen antibody (Ber-EP4), Epithelial Membrane Antigen (EMA, MUC-1, CA 15-3, CD227), Epithelial Sodium Channel-α, Epithelial Sodium Channel-β, Epithelial Sodium Channel-γ, Epithelial Sodium Channel-δ, Epithelium specific antigen (EP-CAM, ESA) (AUA1), Epithelium/endothelial cells [PCX, Podocalyxin], Exo-1 (Pa-G14), EZH2, Ezrin, Fas Ligand/TNFSF6, Fibrinogen (1F3), Foxal, GABRP (ENSMBL ID: ENSG00000094755.17), Galectin-3, GGT (gamma-glutamyl transpeptidase), Glutamine Synthetase, Heat Shock Protein 27 [HSP27], HLA-DR, Lactoferrin, LAMP-1 (lysosomal-associated membrane protein 1), MMR, NCAM-L1 (neural cell adhesion molecule L1), Nectin-2/CD112 (ENSMBL ID: ENSG00000130202.10), Normal Epithelial Cell Specific-1 (NES1)/kallikrein-10, NSE (neuron-specific enolase), Ovarian Cancer Antigen [CA125], P2X7 (ENSMBL ID: ENSG00000089041.17), p63, P-Cadherin, pIgR, Prominin-1 (CD133) (ENSMBL ID: ENSG00000007062.12), Prostasin/Prss8, Prostate Specific Antigen [PSA], Prostatic Binding Protein (PBP), Protein Gene Product 9.5 (PGP 9.5), PSCA (Prostate stem cell antigen), Rab13 (ENSMBL ID: ENSG00000143545.10), RAGE, Rex-1 (zinc-finger protein-42, Zfp42) (ENSMBL ID: ENSG00000179059.10), Secretory Component (SC), Sucrase-isomaltase (SI), Surfactant Protein A, Surfactant Protein B, Surfactant protein C (SPC), Surfactant Protein D, Survivin, TfR (Transferrin Receptor), Transthyretin, UGRP1/SCGB3A2, VAT-1 (ENSMBL ID: ENSG00000108828.16), or Vimentin.

In some embodiments, the fetal epithelial cell marker is a cytokeratin. In some embodiments, the cytokeratin is Human Cytokeratin 1 (CK1), Human Cytokeratin 2 (CK2), Human Cytokeratin 3 (CK3), Human Cytokeratin 4 (CK4) (ENSMBL ID: ENSG00000170477.13), Human Cytokeratin 5 (CK5), Human Cytokeratin 6 (CK6), Human Cytokeratin 7 (CK7) (ENSMBL ID: ENSG00000135480.17), Human Cytokeratin 8 (CK8) (ENSMBL ID: ENSG00000170421.13), Human Cytokeratin 9 (CK9) (ENSMBL ID: ENSG00000171403.10), Human Cytokeratin 10 (CK10) (ENSMBL ID: ENSG00000186395.9), Human Cytokeratin 13 (CK13) (ENSMBL ID: ENSG00000171401.15), Human Cytokeratin 14 (CK14), Human Cytokeratin 15 (CK15) (ENSMBL ID: ENSG00000171346.16), Human Cytokeratin 16 (CK16), Human Cytokeratin 17 (CK17) (ENSMBL ID: ENSG00000128422.18), Human Cytokeratin 18 (CK18) (ENSMBL ID: ENSG00000111057.11), or Human Cytokeratin 19 (CK19) (ENSMBL ID: ENSG00000171345.13). In some embodiments, the binding agent against the epithelial markers is a pan CK binding agent. A pan CK binding agent is a binding agent targeting several cytokeratins at once.

In some embodiments, the fetal cell marker is a fetal endothelial cell marker. In some embodiments, the fetal endothelial cell marker is CD146, CD141, ACE/CD143 (ENSMBL ID: ENSG00000159640.17), C1qR1/CD93 (ENSMBL ID: ENSG00000125810.10), VE-Cadherin, CC Chemokine Receptor D6, CD31/PECAM-1 (ENSMBL ID: ENSG00000261371.6), CD34 (ENSMBL ID: ENSG00000174059.17), CD36/SR-B3, CD144 (ENSMBL ID: ENSG00000179776.19), CD151 (ENSMBL ID: ENSG00000177697.19), CD160 (ENSMBL ID: ENSG00000117281.16), CD300g/Nepmucin, CL-K1/COLEC11 (ENSMBL ID: ENSG00000118004.18), CL-P1/COLEC12 (ENSMBL ID: ENSG00000158270.12), Coagulation Factor III/Tissue Factor, DC-SIGNR/CD299, DCBLD2/ESDN, ECSCR, EMMPRIN/CD147 (ENSMBL ID: ENSG00000172270.22), Endoglin/CD105, Endomucin, Endosialin/CD248 (ENSMBL ID: ENSG00000174807.4), EPCR (ENSMBL ID: ENSG00000101000.6), Erythropoietin R, ESAM (ENSMBL ID: ENSG00000149564.12), FABPS/E-FABP, FABP6, (ENSMBL ID: ENSG00000170231.16), ICAM-1/CD54, ICAM-2/CD102, IL-1 RI, IL-13 R alpha 1, Integrin alpha 4/CD49d, Integrin alpha 4 beta 1, Integrin alpha 4 beta 7/LPAM-1, Integrin beta 2/CD18, KLF4 (ENSMBL ID: ENSG00000136826.15), LYVE-1, MCAM/CD146 (ENSMBL ID: ENSG00000076706.17), Nectin-2/CD112 (ENSMBL ID: ENSG00000130202.10), PD-ECGF/Thymidine Phosphorylase, Podocalyxin, Podoplanin, S1P1/EDG-1, S1P2/EDG-5, S1P3/EDG-3, S1P4/EDG-6, S1P5/EDG-8, E-Selectin/CD62E, E-Selectin (CD62E)/P-Selectin (CD62P), P-Selectin/CD62P, SLAM/CD150, Stabilin-1, Stabilin-2, TEM7/PLXDC1, TEM8/ANTXR1, Thrombomodulin/BDCA-3, THSD1, THSD7A, Tie-2, TNF RI/TNFRSF1A, TNF RII/TNFRSF1B, TRA-1-85/CD147, TRAIL R2/TNFRSF10B, TRAILR1/TNFRSF10A, VCAM-1/CD106 (ENSMBL ID: ENSG00000162692.12), VE-Statin, VEGFR1/Flt-1, VEGFR2/KDR/Flk-1, VEGFR3/Flt-4, VG5Q, or vWF-A2.

In some embodiments, the fetal endothelial cell marker is Endoglin/CD105 (ENSMBL ID: ENSG00000106991.14), CD146 or CD141. In some embodiments, the fetal endothelial cell marker is Endoglin/CD105. In some embodiments, binding agents against the endothelial marker is covalently conjugated by any known method in the art. In some embodiments, fluorescent binding agents against the endothelial marker is covalently conjugated with Alexa Fluor-488. In some embodiments, fluorescent binding agents against the endothelial marker is covalently conjugated with FITC.

In some embodiments, the one or more binding agents is directed against a maternal cell marker. The term maternal cell marker as used herein refers to a nucleic acid or a protein enriched in the maternal cells compared to a reference cell e.g., a fetal cell. In some embodiments, the maternal cell marker is CD45. In some embodiments, maternal cells may be detected using a nucleotide probe specific for a maternal cell mRNA. CD45 is also known as leukocyte common antigen. CD45 is a transmembrane protein expressed by all differentiated hematopoietic cells except erythrocytes and plasma cells. The CD45 protein exists in different forms which are all produced from a single complex gene giving rise to eight different mature mRNAs and resulting in eight different protein products. It is expressed on all leukocytes but not on other cells, and thus functions as a pan-leukocyte marker including the different and diverse types of leukocytes (or white blood cells) such as neutrophils, eosinophils, basophils, lymphocyte (B and T cells), monocytes and macrophages.

In some embodiments, the binding agents are directed to nuclear marker. The term nuclear marker as used herein refers to a nucleic acid or a protein enriched in any nucleus compared to the cytoplasm of a fetal cell or a maternal cell. In some embodiments, the binding agent is any agent that binds or intercalates with nuclear DNA. In some embodiments, the binding agent may be, 4′,6-diamidino-2-phenylindole (DAPI), Propidium iodide, TO-PRO®-3, or Hoechst 33342. In some embodiments, the binding agents are directed to cytoplasmic marker. The term cytoplasmic marker as used herein refers to a nucleic acid or a protein enriched in a cytoplasm compared to the nucleus of a fetal cell or a maternal cell. Non-limiting example of cytoplasmic markers include cytokeratins.

In some embodiments, the binding agent can be directed to one or more cell markers. In some embodiments, the binding agent can be directed to two or more fetal cell markers. In some embodiments, the binding agent can be directed to two or more maternal cell markers. In some embodiments, the binding agent can be directed to a fetal cell marker and a maternal cell marker. In some embodiments, more than one binding agent is used.

Biological Sample

In some embodiments, the biological sample comprises a population of cells, wherein the population of cells comprises at least one of a maternal cell and a fetal cell. In some embodiments, the fetal cell is a fetal trophoblast cell. In some embodiments, the fetal cells may be fetal nucleated red blood cells (fnRBC), fetal lymphocytes (B or T cell), or fetal progenitor (stem) cells. In some embodiments, the fetal cell is a male fetal cell. In some embodiments, the male fetal cells are sorted by any of the methods used herein. In some embodiments, the male fetal cells are sorted by any of the binding agents used herein. In some embodiments, the fetal cell is a female fetal cell. In some embodiments, the female fetal cells are sorted by any of the methods used herein. In some embodiments, the female fetal cells are sorted by any of the binding agents used herein.

In some embodiments, the biological sample is a blood sample or a cervical secretion sample. Generally, cervical samples contain fetal trophoblasts but do not contain other fetal cell types such as fnRBC, lymphocytes, or stem/progenitor cells.

In some embodiments, blood samples from pregnant women are obtained. In general, blood samples are drawn using a syringe or similar device, such as a Vacutainer® tube. The syringe or tube can contain anticoagulants, preservatives, stabilizers, fixatives, and the like to preserve the biological sample in a form suitable for analysis. In some embodiments, the syringe or tube contains an anticoagulant. In some embodiments, the syringe or tube contains EDTA (ethylenediaminetetraacetic acid). In some embodiments, the syringe or tube contains a fixative. In some embodiments, the syringe or tube contains paraformaldehyde. In some embodiments, the syringe or tube contains EDTA and paraformaldehyde.

The amount of blood drawn can be from about 0.5 mL to about 40 mL. In some embodiments, the amount of blood drawn is about 0.5, 0.6. 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mL. In some embodiments, the amount is about 10 mL. In some embodiments, the amount is about 20 mL. In some embodiments, the amount is about 30 mL. In some embodiments, the amount is about 40 mL.

The biological sample is optionally fixed with a suitable fixative to preserve the cells. Suitable fixatives include formaldehyde (or paraformaldehyde), glutaraldehyde, methanol, acetone, mixtures thereof, and the like. The fixative can be provided as a solution, for example in phosphate-buffered saline (PBS), and the like, or as a solid, for example spray-dried on the walls of a collecting container. The osmolarity of the solution used is generally sufficiently close to normal physiological osmolarity that cells are not ruptured prior to fixation. The amount of fixative used is an amount sufficient to fix the biological sample. In some embodiments, the fixative solution is 5% paraformaldehyde in PBS. In some embodiments, the amount of fixative solution is about 0.01, 0.05, 0.10, 0.15, 0.20, 0.25, 0.3, 0.35, 0.4, 0.45, 0.50, 0.55, 0.60, 0.65, 0.70, 0.75, 0.80, 0.85, 0.90, 1.0, 1.25, 1.50, or 2.0 times the volume of the biological sample. In some embodiments, the cells are washed after fixation.

In some embodiments, the fixation is performed on a non-enriched biological sample. In some embodiments, the fixation is performed on a non-enriched blood sample. Fixation may be in the blood tube or may only be added after blood is partially processed. The samples may be shipped for testing. In some embodiments, the sample shipments may reach the testing site 18-72 h after the blood is drawn. In some embodiments, the biological sample is contacted with the fixation solution no more than 24 hours after the biological sample has been provided. In some embodiments, the biological sample is contacted with the fixation solution no more than 18, 19, 20, 21, 22, 23, 24 hours after the biological sample has been provided. In some embodiments, the biological sample is contacted with the fixation solution no more than 12 hours, such as 8 hours, 4 hours, 2 hours, 1 hour, 30 minutes, 15 minutes after the biological sample has been provided.

In some embodiments, the biological sample is optionally processed to remove RBCs. RBCs can be removed by standard methods, for example by binding to anti-glycophorin-A antibodies, which can further be bound to a solid support for separation, or can simply aggregate RBCs from suspension.

In some embodiments, the cells (particularly maternal red blood cells which have no DNA and are not of interest) are lysed using chemical and/or mechanical lysis. In some embodiments, the chemical lysis comprises a lysis buffer comprising a protease inhibitor, phosphate buffered saline and Triton X100. In some embodiments, the cells can be frozen after the addition of the lysis buffer at −80° C. for about 30 minutes to about 72 hours. Alternatively, the cell lysate may be stored in a range of 2 to 8° C. or at room temperature. In some embodiments, the cells are centrifuged, and the unlysed cells are collected. In some embodiments, this is performed by spinning the cells in a centrifuge at room temperature.

RBCs can also be removed by selective lysis. In some embodiments, the RBCs are lysed using ammonium chloride or a hemolytic surfactant (for e.g., M. Manaargadoo-Catin et al., Adv Colloid Interface Sci (2016) 228:1-16). In some embodiments, the RBCs are removed by contact with a hemolytic surfactant. In some embodiments, the surfactant is a polyoxyethylene surfactant. In some embodiments, the surfactant is Triton X-100®. In some embodiments, the surfactant further comprises a solution. In some embodiments, the surfactant is a polyoxyethylene surfactant solution or suspension. In some embodiments, the concentration of the solution is selected to lyse maternal RBCs without affecting fetal cells. In some embodiments, the biological sample comprises a cellular fraction obtained from the blood sample.

In some embodiments, following pre-treatment to reduce or eliminate the maternal RBCs, the remaining biological sample is washed to remove or reduce the amount of surfactant and fixative that may remain. Conventional methods for cell and molecular biology can be employed. For example, without limitation, the biological sample can be diluted in PBS, which may optionally contain bovine serum albumin (BSA), and then centrifuged. The resulting pellet can be further washed, and the supernatants can be removed by aspiration.

In some embodiments, whole blood protocol is performed to provide a population of cells from whole blood (without removing RBCs) that is substantially enriched in trophoblasts, and optionally stained to distinguish between trophoblasts and maternal cells in the biological sample. The cells can be substantially enriched in trophoblasts as shown in the methods provided in the disclosure.

In some embodiments, the biological sample comprises cervical secretion. Cervical secretions can be collected by using an endocervical brush and other methods (for e.g., A. N. Imudia et al. Fertil. Steril. (2010) 96(6):1725-30; G. Moser et al., Hum. Repro. Update (2018) 24(4):484-96; C. V. Jain et al., Sci. Transl. Med. (2016) 8:363re4). Cervical secretion samples also contain maternal cells such as squamous cells, and may contain additional debris, such as blood elements, spermatozoa, mucus, and particulate contaminants. Mucus can be lysed using known agents, for example mucolytic agents like L-acetyl cysteine, and/or enzymes such as liberase blendzyme (for e.g., M. G. Katz-Jaffe et al., BIOG. (2005) 112:595-600). Cervical secretion samples can be washed using conventional methods for molecular biology. For example, without limitation, the biological sample can be diluted in PBS, which may optionally contain bovine serum albumin (BSA), and then centrifuged. The resulting pellet can be further washed, and the supernatants removed by aspiration.

In some embodiments, the biological sample is then enriched in fetal cells (trophoblasts) following the pre-treatment described above. In some embodiments, enrichment results in a population of cells in which the fetal cells are present in a ratio of fetal cell to other cells of at least about 1:100, at least about 1:90, at least about 1:80, at least about 1:70, at least about 1:60, at least about 1:50, at least about 1:40, at least about 1:30, at least about 1:20, at least about 1:10, at least about 1:5, about 1:1, greater than about 1:1, greater than about 5:1, greater than about 10:1, or greater than about 100:1. In some embodiments, the ratio of fetal cell to other cell is between about 1:1,000 and about 100:1. In some embodiments, the ratio of fetal cell to other cell is between about 1:100 and about 50:1. In some embodiments, the ratio of fetal cell to other cell is between about 1:10 and about 10:1. In some embodiments, the ratio of fetal cell to other cell is about 1:1.

Labels

In some embodiments, one or more binding agents is bound to a label. The term label as used herein refers to any compound, molecule or radioactive moiety known in the art that can be used to provide a detectable and/or quantifiable effect. The label can be attached to a binding agent. In some embodiments, the label can be attached to a nucleic acid or protein (e.g., antibody, antibody fragment, and/or enzyme). In some embodiments, the label can be attached to a probe system. In some embodiments, the label can be attached to an antibody or a fragment thereof.

In some embodiments, the label can be attached to the binding agent by a covalent bond. In some embodiments, the label can be attached to the binding agent by a non-covalent bond. In some embodiments, the label can be attached to the binding agent by a linker. As used herein, the linker can be any functional group (e.g., a chemical) which connects a label to the binding agent. In some embodiments, more than one linker can be used. In some embodiments, the label is bound covalently or non-covalently to the one or more binding agents. In some embodiments, the label is bound covalently to the one or more binding agents. In some embodiments, the label is bound non-covalently to the one or more binding agents.

In some embodiments, the label is a dye, a radiolabel, a hapten, a luminogenic, a phosphorescent or a fluorogenic moiety, or a mass tag. In some embodiments, the label is a dye and wherein the dye is a fluorescent dye. In some embodiments, the fluorescent dye is a xanthene dye, a coumarin dye, a pyrene dye or a cyanine dye.

In some embodiments, the fluorescent dye is Indo-1, Ca saturated, Indo-1 Ca2+, Cascade Blue BSA pH 7.0, Cascade Blue, LysoTracker Blue, Alexa 405, LysoSensor Blue pH 5.0, LysoSensor Blue, DyLight 405, DyLight 350, BFP (Blue Fluorescent Protein), Alexa 350, 7-Amino-4-methylcoumarin pH 7.0, Amino Coumarin, AMCA conjugate, Coumarin, 7-Hydroxy-4-methylcoumarin, 7-Hydroxy-4-methylcoumarin pH 9.0, 6,8-Difluoro-7-hydroxy-4-methylcoumarin pH 9.0, Hoechst 33342, Pacific Blue, Hoechst 33258, Hoechst 33258-DNA, Pacific Blue antibody conjugate pH 8.0, PO-PRO-1, PO-PRO-1-DNA, POPO-1, POPO-1-DNA, DAPI-DNA, DAPI, Marina Blue, SYTOX Blue-DNA, CFP (Cyan Fluorescent Protein), eCFP (Enhanced Cyan Fluorescent Protein), 1-Anilinonaphthalene-8-sulfonic acid (1,8-ANS), Indo-1, Ca free, 1,8-ANS (1-Anilinonaphthalene-8-sulfonic acid), BO-PRO-1-DNA, BOPRO-1, -DNA, SYTO 45-DNA, evoglow-Pp1, evoglow-Bs1, evoglow-Bs2, Auramine O, DiO, LysoSensor Green pH 5.0, Cy 2, LysoSensor Green, Fura-2, high Ca, Fura-2 Ca2+sup>, SYTO 13-DNA, YO-PRO-1-DNA, YOYO-1-DNA, eGFP (Enhanced Green Fluorescent Protein), LysoTracker Green, GFP (S65T), BODIPY FL, Sapphire, BODIPY FL conjugate, MitoTracker Green, MitoTracker Green FM, Fluorescein 0.1 M NaOH, Calcein pH 9.0, Fluorescein pH 9.0, Calcein, Fura-2, no Ca, Fluo-4, FDA, DTAF, Fluorescein, Fluorescein antibody conjugate pH 8.0, CFDA, FITC, Alexa Fluor 488 hydrazide-water, DyLight 488, 5-FAM pH 9.0, FITC antibody conjugate pH 8.0, Alexa 488, Rhodamine 110, Rhodamine 110 pH 7.0, Acridine Orange, Alexa Fluor 488 antibody conjugate pH 8.0, BCECF pH 5.5, PicoGreendsDNA quantitation reagent, SYBR Green I, Rhodaminen Green pH 7.0, CyQUANT GR-DNA, NeuroTrace 500/525, green fluorescent Nissl stain-RNA, DansylCadaverine, Rhodol Green antibody conjugate pH 8.0, Fluoro-Emerald, Nissl, Fluorescein dextran pH 8.0, Rhodamine Green, 5-(and-6)-Carboxy-2′,7′-dichlorofluorescein pH 9.0, DansylCadaverine, eYFP (Enhanced Yellow Fluorescent Protein), Oregon Green 488, Oregon Green 488 antibody conjugate pH 8.0, Fluo-3, BCECF pH 9.0, SBFI-Na+, Fluo-3 Ca2+, Rhodamine 123, FlAsH, Calcium Green-1 Ca2+, Magnesium Green, DM-NERF pH 4.0, Calcium Green, Citrine, LysoSensor Yellow pH 9.0, TO-PRO-1-DNA, Magnesium Green Mg2+, Sodium Green Na+, TOTO-1-DNA, Oregon Green 514, Oregon Green 514 antibody conjugate pH 8.0, NBD-X, DM-NERF pH 7.0, NBD-X, CI-NERF pH 6.0, Alexa 430, Alexa Fluor 430 antibody conjugate pH 7.2, CI-NERF pH 2.5, Lucifer Yellow, CH, LysoSensor Yellow pH 3.0, 6-TET, SE pH 9.0, Eosin antibody conjugate pH 8.0, Eosin, 6-Carboxyrhodamine 6G pH 7.0, 6-Carboxyrhodamine 6G, hydrochloride, Bodipy R6G SE, BODIPY R6G, 6 JOE, Cascade Yellow antibody conjugate pH 8.0, Cascade Yellow, mBanana, Alexa Fluor 532 antibody conjugate pH 7.2, Alexa 532, Erythrosin-5-isothiocyanate pH 9.0, 6-HEX, SE pH 9.0, mOrange, mHoneydew, Cy 3, Rhodamine B, DiI, 5-TAMRA-MeOH, Alexa 555, Alexa Fluor 555 antibody conjugate pH 7.2, DyLight 549, BODIPY TMR-X, SE, BODIPY TMR-X, PO-PRO-3-DNA, PO-PRO-3, Rhodamine, Bodipy TMR-X conjugate, POPO-3, Alexa 546, BODIPY TMR-X antibody conjugate pH 7.2, Calcium Orange Ca2+, TRITC, Calcium Orange, Rhodaminephalloidin pH 7.0, MitoTracker Orange, MitoTracker Orange, Phycoerythrin, Magnesium Orange, R-Phycoerythrin pH 7.5, 5-TAMRA pH 7.0, 5-TAMRA, Rhod-2, FM 1-43, Rhod-2 Ca2+, Tetramethylrhodamine antibody conjugate pH 8.0, FM 1-43 lipid, LOLO-1-DNA, dTomato, DsRed, Dapoxyl (2-aminoethyl) sulfonamide, Tetramethylrhodamine dextran pH 7.0, Fluor-Ruby, Resorufin, Resorufin pH 9.0, mTangerine, LysoTracker Red, Lissaminerhodamine, Cy 3.5, Rhodamine Red-X antibody conjugate pH 8.0, Sulforhodamine 101, JC-1 pH 8.2, JC-1, mStrawberry, MitoTracker Red, MitoTracker Red, X-Rhod-1 Ca2+, Alexa Fluor 568 antibody conjugate pH 7.2, Alexa 568, 5-ROX pH 7.0, 5-ROX (5-Carboxy-X-rhodamine, triethylammonium salt), BO-PRO-3-DNA, BOPRO-3, BOBO-3-DNA, Ethidium Bromide, ReAsH, Calcium Crimson, Calcium Crimson Ca2+, mRFP, mCherry, Texas Red-X antibody conjugate pH 7.2, HcRed, DyLight 594, Ethidium homodimer-1-DNA, Ethidiumhomodimer, Propidium Iodide, SYPRO Ruby, Propidium Iodide-DNA, Alexa 594, BODIPY TR-X, SE, BODIPY TR-X, BODIPY TR-X phallacidin pH 7.0, Alexa Fluor 610 R-phycoerythrin streptavidin pH 7.2, YO-PRO-3-DNA, Di-8 ANEPPS, Di-8-ANEPPS-lipid, YOYO-3-DNA, Nile Red-lipid, Nile Red, DyLight 633, mPlum, TO-PRO-3-DNA, DDAO pH 9.0, Fura Red, high Ca, Allophycocyanin pH 7.5, APC (allophycocyanin), Nile Blue, TOTO-3-DNA, Cy 5, BODIPY 650/665-X, Alexa Fluor 647 R-phycoerythrin streptavidin pH 7.2, DyLight 649, Alexa Fluor 647 antibody conjugate pH 7.2, Alexa 647, Fura Red Ca2+, Atto 647, Fura Red, low Ca, Carboxynaphthofluorescein pH 10.0, Alexa 660, Alexa Fluor 660 antibody conjugate pH 7.2, Cy 5.5, Alexa Fluor 680 antibody conjugate pH 7.2, Alexa 680, DyLight 680, Alexa Fluor 700 antibody conjugate pH 7.2, Alexa 700, FM 4-64, 2% CHAPS, or FM 4-64.

The sorted/enriched cells can be verified as fetal cells by genetic analysis. In some embodiments, the fetal cells obtained by any of the methods of the disclosure can be used to identify genetic abnormalities in the fetal cells.

In some embodiments, the enriched cell(s) is not isolated from the biological sample but rather detection is performed on the cell(s) while still present in the biological sample. The biological sample may be present on a glass slide and the detection may be performed using microscopy or lasers.

In some embodiments, the disclosure provides a method of detecting fetal cells in a biological sample, said method comprising the steps of (a) contacting the biological sample with one or more binding agents directed against at least one of the group consisting of a nuclear marker, a fetal cell marker, or a maternal cell marker, (b) visualizing the cells of the biological sample based on detection of the one or more binding agents bound to said at least one nuclear marker, a fetal cell marker, or a maternal cell marker; and (c) determining the presence of fetal cells as those bound by the binding agents from step (b).

In some embodiments, the cells are visualized under a microscope or a laser. In some embodiments, the cells are visualized under a microscope.

In some embodiments, the fetal cells are processed for downstream whole genome amplification and sequencing. In some embodiments, the fetal cells can be stained with fetal cell-specific antibodies conjugated to a dye. In some embodiments, the dye may be fluorescein isothiocyanate (FITC).

Generally, the maternal RBCs are lysed early on to get rid of them. Once single or near single fetal cells are obtained, the cells are lysed toward DNA analysis. In some embodiments, nucleic acids are obtained from a cell population enriched in fetal cells by lysis. In some embodiments the fetal cells are lysed in individual pools. In some embodiments multiple fetal cells are lysed in the same pool if they were isolated as a clump of from about 2 to about 20 fetal cells. Alternatively, the cell population can be sorted by known techniques such as FACS or using a microfluidic device, for example without limitation, a Namocell Namo™, Pala™ or Hana™ single cell dispenser (Namocell Inc., Mountain View, CA) or submitted directly into a sorting/input device for single-cell sequencing.

### II. Prenatal Screening

In some embodiments, the fetal cells obtained by any of the methods described herein can be further used for identifying genetic or epigenetic abnormalities or variations in the cells. In some embodiments, the disclosure provides a method for genotyping a fetus, the method comprising: (a) isolating fetal cells according to the methods described herein; (b) lysing the fetal cells to obtain the fetal nucleic acid; (c) amplifying the fetal cell nucleic acid or a portion thereof; and (d) genotyping the fetus by evaluating for a genetic difference compared to a maternal cell.

Nucleic acids obtained by the methods herein are examined for indications of a genetic difference. All nucleic acids or only portions thereof can be examined. In some embodiments, the nucleic acids are amplified before examination.

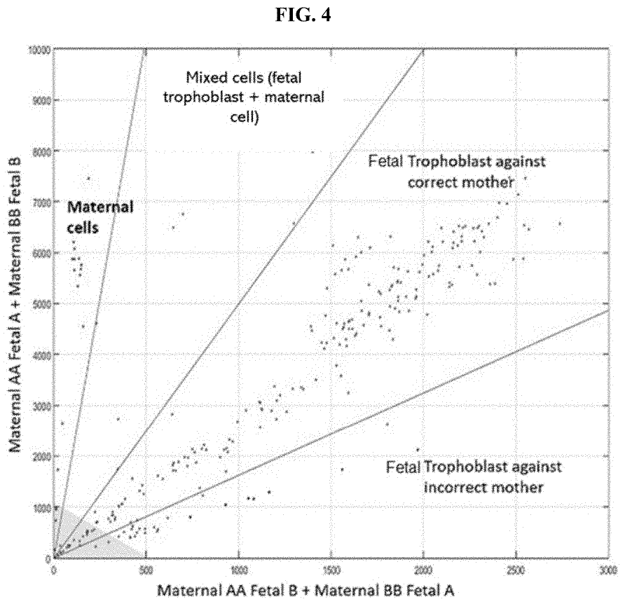

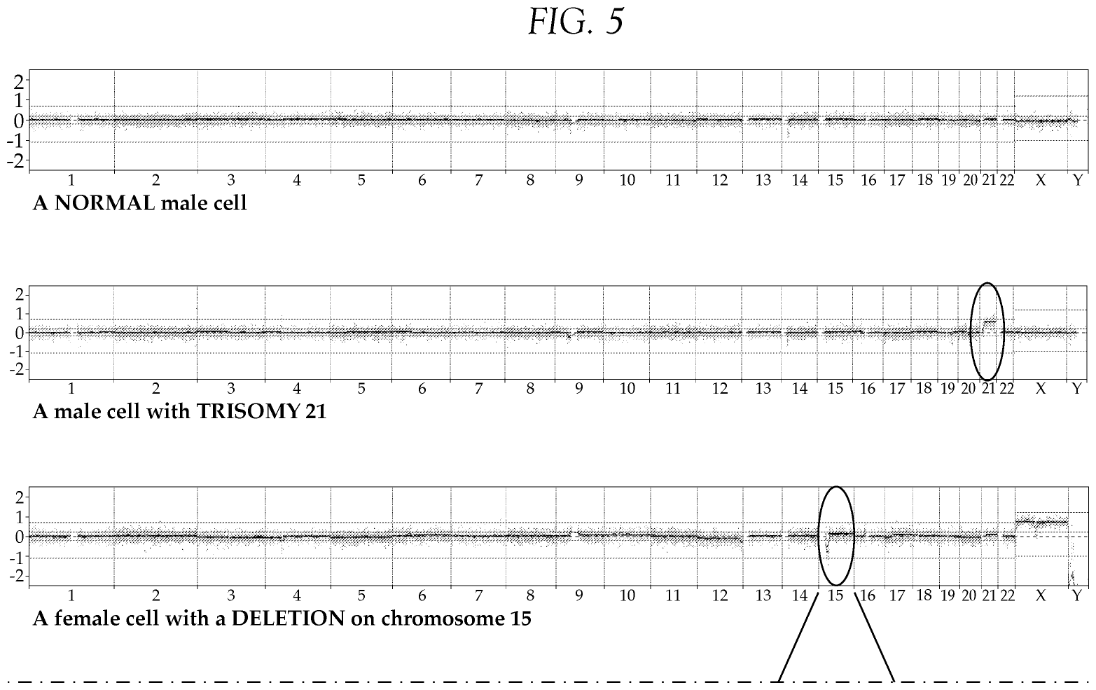

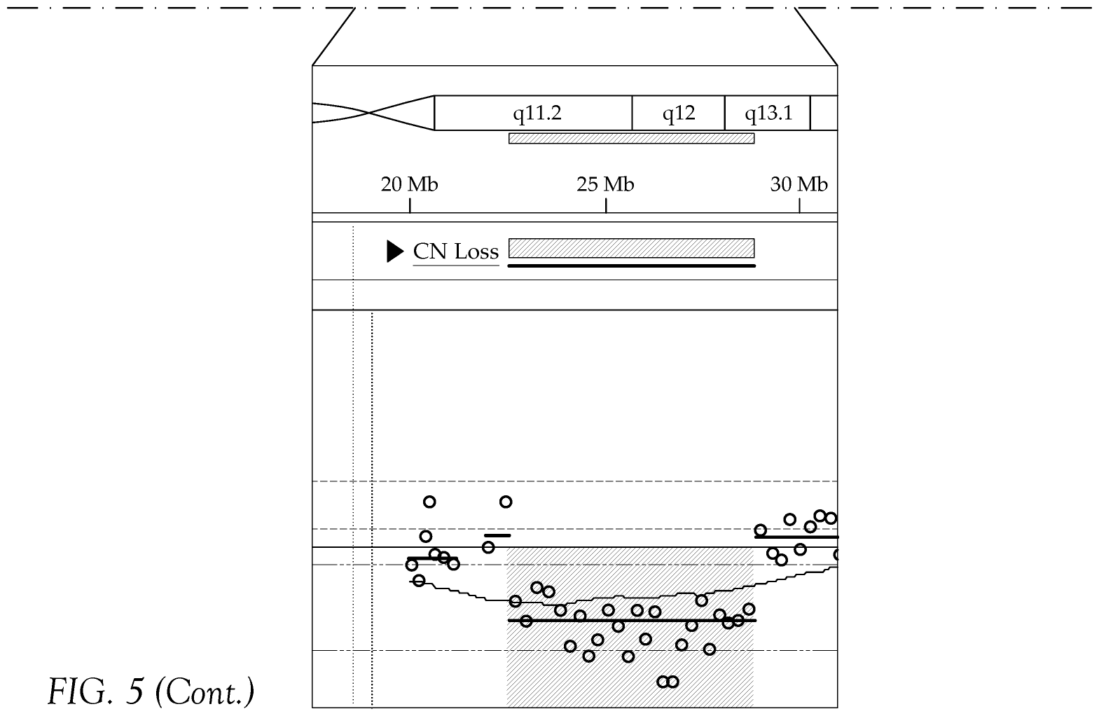

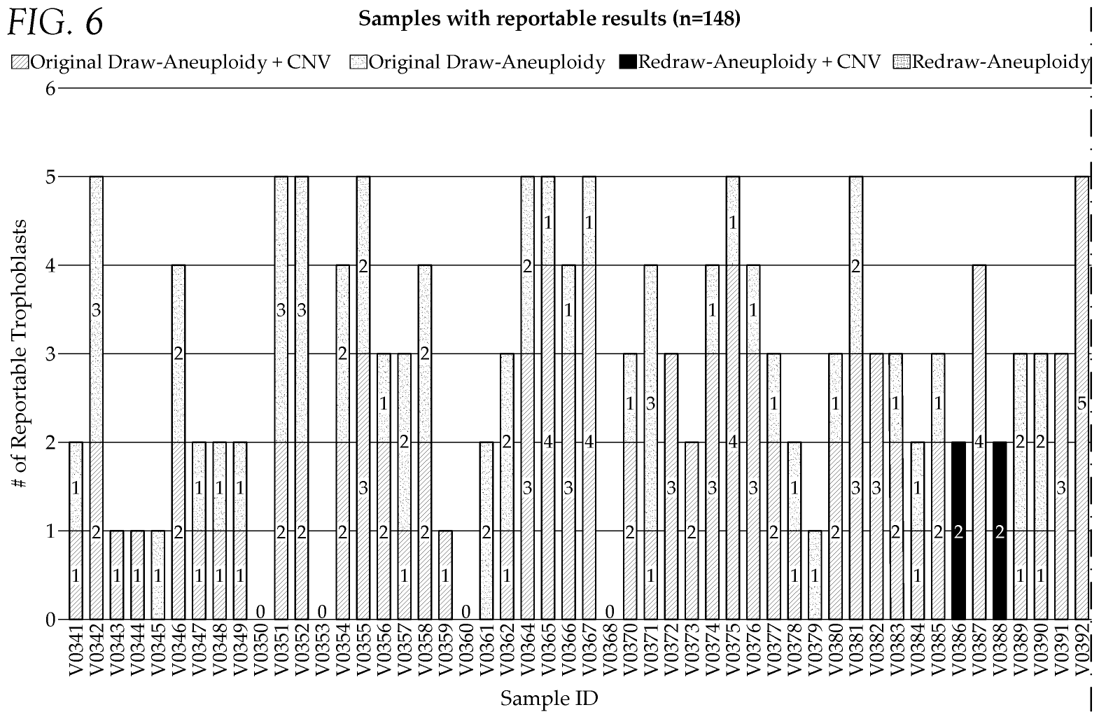

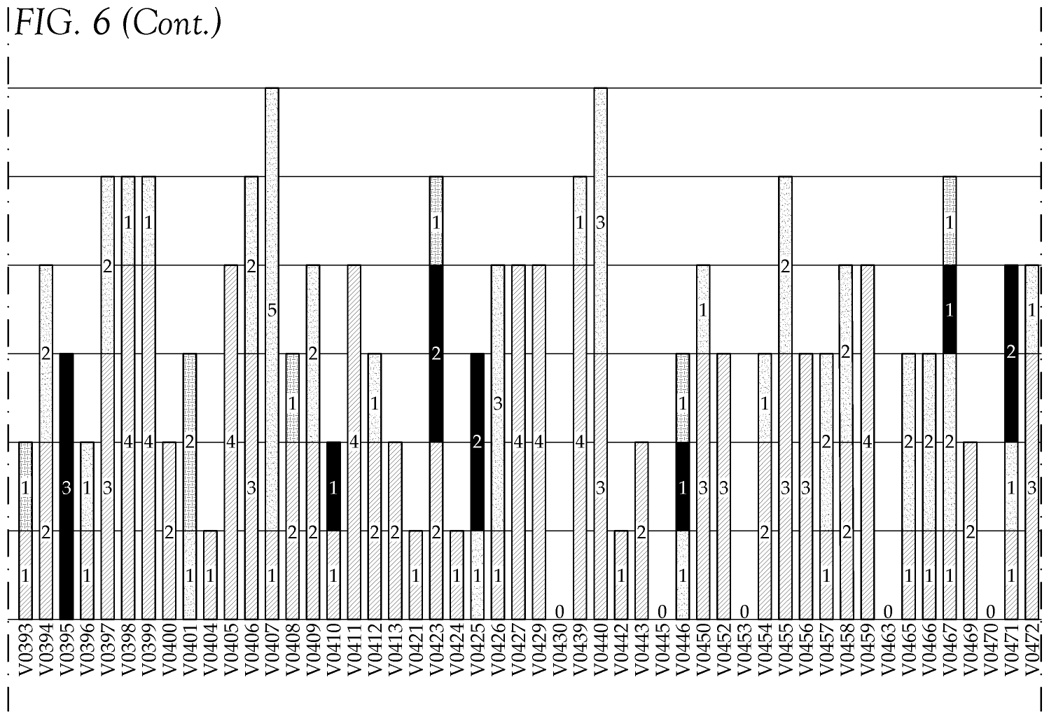

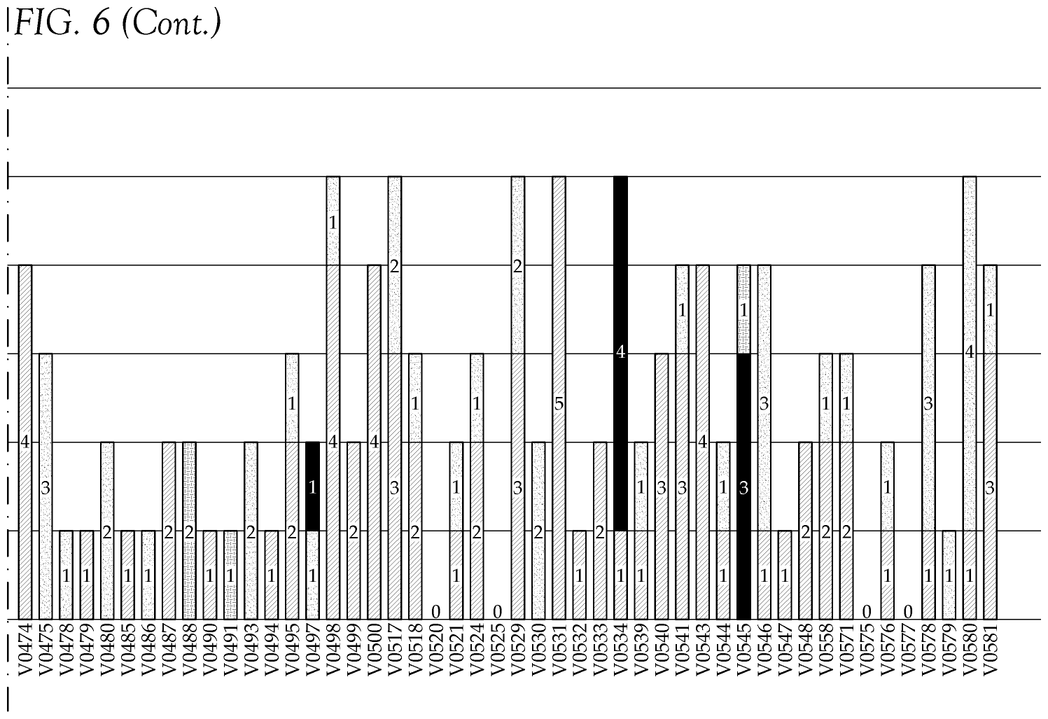

In general, the genetic difference is a difference between the nucleic acids in the fetal cell, their sequence, organization, gene or fragment copy number, and the like compared to the maternal cell. Low coverage SNP data from each cell acquired by next-generation sequencing (NGS) is generally compared against the results of a high-density SNP array performed on the mother's genomic DNA. As shown in FIG. 4, cells were classified based on comparison of maternal vs. fetal SNP data and established thresholds. A fetal cell has hundreds to thousands of alleles which are not present in the mother as these alleles are inherited from the father, hence classifying it as fetal and not maternal.

For example, without limitation, the difference can be a copy number variation (CNV) such as the duplication or absence of an entire chromosome (aneuploidy), the duplication or absence of a part of a chromosome (partial aneuploidy), and the duplication or absence of one or more genes or parts of genes. The difference can also be a polymorphism, a translocation between two chromosomes, an indel (insertion or deletion), or a single nucleotide variant (SNV). Differences further include the presence of two or more sets of chromosomes, indicating multiple fetuses (e,g., twins, triplets, and the like). Differences also include genotypes wherein a pair of chromosomes consists of two identical or partially identical copies, which can indicate uniparental disomy. Differences further include mosaicism, such as confined placental mosaicism, and alleles that are associated with pathological conditions or genetic disorders.

In some embodiments, the genetic abnormalities may include deletions and trisomy. In some embodiments, the genetic abnormality is trisomy 21. In some embodiments, the genetic abnormality is a deletion on chromosome 15. In some embodiments, the genetic variation may include a haplotype that may carry the risk of diabetes.

Epigenetic variations are defined as stable and heritable alterations in gene expression and cellular function without changes to the original DNA sequence. In some embodiments, the epigenetic variation is DNA methylation. The term “methylation” refers to cytosine methylation at positions C5 or N4 of cytosine, the N6 position of adenine or other types of nucleic acid methylation. In some embodiments, the epigenetic variations may include an SNP associated with IQ or low cancer risk.

In some embodiments, the pathological condition is lp36 deletion syndrome, 18p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, aceruloplasminemia, acheiropodia, achondrogenesis type II, achondroplasia, acute intermittent porphyria, adenylosuccinate lyase deficiency, adrenoleukodystrophy, alagille syndrome, ADULT syndrome, Aicardi-Goutieres syndrome, Albinism, Alexander disease, alkaptonuria, alport syndrome, alternating hemiplegia of childhood, amyotrophic lateral sclerosis, frontotemporal dementia, alstrom syndrome, amelogenesis imperfecta, aminolevulinic acid dehydratase deficiency porphyria, androgen insensitivity syndrome, angelman syndrome, apert syndrome, arthrogryposis-renal dysfunction-cholestasis syndrome, ataxia telangiectasia, axenfeld syndrome, Beare-Stevenson cutis gyrata syndrome, Beckwith-Wiedemann syndrome, Benjamin syndrome, biotinidase deficiency, Bjornstad syndrome, bloom syndrome, Birt-Hogg-Dube syndrome, brody myopathy, brunner syndrome, CADASIL syndrome, CARASIL syndrome, Chronic granulomatous disorder, campomelic dysplasia, canavan disease, carpenter syndrome, cerebral dysgenesis-neuropathy-ichthyosis-keratoderma syndrome (SEDNIK), cystic fibrosis, charcot-marie-tooth disease, CHARGE syndrome, Chediak-Higashi syndrome, cleidocranial dysostosis, cockayne syndrome, Coffin-Lowry syndrome, Cohen syndrome, collagenopathy, types II and XI, congenital insensitivity to pain with anhidrosis (CIPA), congenital muscular dystrophy, Cornelia de Lange syndrome (CDLS), cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (genetic hypercalciuria), Denys-Drash syndrome, De Grouchy syndrome, down syndrome, Di George's syndrome, Distal hereditary motor neuropathies, multiple types, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Straussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, familial, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type I (Wermer's syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Trisomy 8, Trisomy 9, Trisomy, 22, Tuberous sclerosis complex (TSC), Turner syndrome, Usher syndrome, Variegate porphyria, von Hippel-Lindau disease, Waardenburg syndrome, Weissenbacher-Zweymuller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xpl 1.2 duplication syndrome, X-linked severe combined immunodeficiency (X-SCID), X-linked sideroblastic anemia (XLSA), 47, XXX (triple X syndrome), XXXX syndrome (48, XXXX), XXXXX syndrome (49, XXXXX), XYY syndrome (47,XYY), or Zellweger syndrome.

In some embodiments, the nucleic acids can be examined by methods known in the art for comparing nucleic acid sequences and/or quantities to a control sample. In some embodiments, the method is quantitative polymerase chain reaction amplification (qPCR), array comparative genomic hybridization (array CGH), SNP arrays, or next generation sequencing (NGS). In some embodiments, commercially available sequencing equipment can be used for genotyping.

It will be apparent to those skilled in the art that a number of different sequencing methods and variations can be used. One method that can be used involves paired end sequencing. Fluorescently labeled sequencing primers can be used to simultaneously sequence both strands of a dsDNA template, as described e.g., by S, Wiemann et al., Anal Biochem (1995) 224:117-21; S. Wiemann et al., Anal Biochem (1996) 234:166-74. This technique has demonstrated multiplex co-sequencing using the four-color dye terminator reaction chemistry pioneered by Prober et al., Science (1987) 238:336.

Comparative Genome Hybridization (CGH) is based on a quantitative two-color fluorescence in situ hybridization (FISH) on metaphase chromosomes. In this method, a test DNA (for example, DNA extracted from a trophoblast) is labeled in one color (for example, green) and mixed in a 1:1 ratio with a reference DNA (e.g., DNA extracted from a control cell) which is labeled in a different color (e.g., red), and the fluorescence is measured. Briefly, genomic DNA is amplified using a degenerate oligonucleotide primer (see for example, D. Wells et al., Nucleic Acids Res. (1999) 27:1214-8), and the amplified DNA is labeled using, for example without limitation, Spectrum Green-dUTP (for the test DNA) or Spectrum Red-dUTP (for the reference DNA). The mixture of labeled DNA samples is precipitated with Cot 1 DNA (Gibco-BRL) and resuspended in a hybridization mixture containing, for example, 50% formamide, 2×SSC, pH 7, and 10% dextran sulfate. Prior to hybridization, the labeled DNA samples (i.e., the probes) are denatured for 10 minutes at 75° C., and allowed to cool at room temperature for 2 minutes. Likewise, the metaphase chromosome spreads are denatured using standard protocols (e.g., dehydration in ethanol, denaturation for 5 minutes at 75° C. in 70% formamide and 2×SSC). Hybridization conditions include incubation at 37° C. for 25-30 hours in a humidified chamber, following by washes in 2×SSC and dehydration using an ethanol series (see, e.g., D. Wells et al., Fertil. Steril. (2002) 78:543-49). The hybridization signal is detected using a fluorescence microscope, and the ratio of the green-to-red fluorescence can be determined using e.g., the Applied Imaging (Santa Clara, Calif.) computer software. If both genomes are equally represented in the metaphase chromosomes (i.e., no deletions, duplication or insertions in the DNA derived from the trophoblast cell), the labeling on the metaphase chromosomes is yellow or orange. Regions which are either deleted or duplicated in the trophoblast cell are stained red or green respectively.

DNA array-based comparative genomic hybridization (CGH-array) is a modified version of CGH and is based on the hybridization of a 1:1 mixture of the test and reference DNA probes on an array containing chromosome-specific DNA libraries (D. G. Hu et al., Mal. Hum. Reprod. (2004) 10:283-89). Methods of preparing chromosome-specific DNA libraries are known in the art (see, for example, A. Balzer et al., Cytogenet. Cell Genet. (1999) 84:233-40). Briefly, single chromosomes are obtained by microdissection or flow-sorting, and the genomic DNA of each of the isolated chromosomes is PCR-amplified using a degenerate oligonucleotide primer.

SNP array is a type of DNA microarray used to detect polymorphisms within a population. A variation at a single site in DNA, single nucleotide polymorphism (SNP) is the most frequent type of variation in the genome. By using SNP array method, the presence of SNPs contained in the sample DNA are analyzed by arranging and attaching several hundred to several hundred thousand biomolecules as a probe, such as DNA, DNA fragment, cDNA, oligonucleotide, RNA or RNA fragment having the known sequences, which are immobilized at intervals on a small solid substrate formed of glass, silicon or nylon. Hybridization occurs between nucleic acids contained in the sample and probes immobilized on the surface, depending on the degree of complementarity. By detecting and interpreting the hybridization, information on the materials contained in the sample can be concurrently obtained.

NGS sequencing results are analyzed using techniques and software tools known in the art. For example, sequence alignments can be obtained using algorithms such as BWA-MEM in Burrows-Wheeler Aligner. Coverage counts can be obtained using software such as bedtools. Data can also be analyzed using software such as NxClinical (BioDiscovery, El Segundo, CA).

### III. Kits and Devices

The present disclosure provides a variety of kits for conveniently and/or effectively carrying out methods of the present disclosure. In some embodiments, the kit comprises reagents suitable for the practice of the methods described herein. Typically, kits will comprise sufficient amounts and/or numbers of components to allow a user to perform multiple experiments.

In some embodiments, the present disclosure provides kits for detecting and/or isolating fetal cells of the present disclosure. In some embodiments, the kit further comprises a stain or a label for visualizing and/or identifying fetal cells.

In some embodiments, the present disclosure provides kits for detecting and/or isolating fetal cell specific nucleic acids of the present disclosure. In some embodiments, the kit may comprise probes that bind to fetal cell specific nucleic acids. In some embodiments, the kit may comprise nucleic acid sequences that bind to fetal cell specific nucleic acids.

In some embodiments, the present disclosure provides kits comprising microbubbles described herein for detecting fetal cells of the present disclosure. In some embodiments, the present disclosure provides kits comprising binding agent for detecting fetal cells of the present disclosure. In some embodiments, the present disclosure provides kits comprising binding agent and microbubbles for detecting fetal cells of the present disclosure.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of the compositions described herein in the buffer solution over a period of time and/or under a variety of conditions.

The present disclosure provides for devices which may incorporate the compositions of the present disclosure. Non-limiting examples of the devices include a microfluidic device, flow cytometer, FACS, microscope, pump, a needle, and the like.

Fetal cells disclosed herein may be detected using various assays known in the art. As used herein, the term “assay” refers to the sequence of activities associated with a reported result, which can include, but is not limited to: cell seeding, preparation of the test material, infection, lysis, analysis, and calculation of results.

In some embodiments, the assay surfaces are sterile and are suitable for culturing cells under conditions representative of the culture conditions during large-scale (e.g., industrial scale) production of the biological product. In some embodiments, the exterior comprises wells, indentations, demarcations, or the like at positions corresponding to the assay surfaces. In some embodiments, the wells, indentations, demarcations, or the like retain fluid, such as cell culture media, over the assay surfaces.

The methods described herein can be performed by utilizing any of a wide range cell assay formats, including, but not limited to cell plates, e.g., 24-well plates, 48-well plates, 96-well plates, or 384-well plates, individual cell culture plates, or flasks, for example T-flasks or shaker flasks.

In some embodiments, the material comprises a microarray plate, a biochip, or the like which allows for the high-throughput, automated testing of a range of test agents, conditions, and/or combinations thereof on the production of a biological product by cultured cells. For example, the material may comprise a 2-dimensional microarray plate or biochip having m columns and n rows of assay surfaces (e.g., residing within wells) which allow for the testing of m×n combinations of test agents and/or conditions (e.g., on a 24-, 96- or 384-well microarray plate). The microarray materials are preferably designed such that all necessary positive and negative controls can be carried out in parallel with testing of the agents and/or conditions.

### IV. Definitions

As used herein, the term “antibody” is referred to in the broadest sense and specifically covers various embodiments, including, but not limited to, polyclonal antibodies, monoclonal antibodies, multispecific antibodies (e.g., bispecific antibodies or trispecific antibodies), human antibodies, humanized antibodies, chimeric antibodies, single chain antibodies, single chain Fv (scFv) formats, diabodies, intrabodies, unibodies, maxibodies, chimeric antigen receptors (CARs), and antibody fragments. As used herein the term “antibody fragment” refers to a portion of a whole antibody or a fusion protein that includes such a portion. Antibody fragments may include antigen binding regions. In some embodiments, antibody fragments include, but are not limited to Fab fragments, Fab′ fragments, F(ab′)₂ fragments, Fv fragments, Fc fragments, variable domains, constant domains, heavy chains, and light chains. Antibodies are primarily amino-acid based molecules but may also include one or more modifications (including, but not limited to sugar moieties, fluorescent labels, chemical tags, etc.).

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “assay” refers to the sequence of activities associated with a reported result, which can include, but is not limited to: cell seeding, preparation of the test material, infection, lysis, analysis, and calculation of results.

The term “binding agent” as used herein refers to a polypeptide, nucleic acid or a small molecule or a large molecule which can interact with a particular cell or cell-compartment thereof. In some embodiments, the cell may be a maternal or fetal cell. In some embodiments, the binding agent may be any antibody, antibody fragment, a nucleic acid (e.g., probe), aptamer, or a small molecule.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “fetal cell” includes cells of direct fetal origin, such as fetal nucleated red blood cells, and other cells derived or descended from the zygote, such as cells of placental or other origin, for example trophoblasts.

The term “fetal cell marker” as used herein refers to a nucleic acid, a protein, an oligosaccharide or a glycoprotein enriched in the fetal cells compared to a reference cell e.g., a maternal cell. In some embodiments, the fetal cell specific RNA sequence may be used as the fetal cell marker.

A “fragment,” as used herein, refers to a portion. For example, fragments of proteins may comprise polypeptides obtained by digesting full-length protein isolated from cultured cells. Fragments of oligonucleotides may comprise nucleotides, or regions of nucleotides.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays, or complementation, e.g., genetic, or biochemical. See, for example, Fields et al. (1989) Nature 340:245-246; U.S. Pat. No. 5,585,245 and PCT WO 98/44350.

As used herein, the term “gene” refers to a nucleic acid sequence that comprises control and most often coding sequences necessary for producing a polypeptide or precursor. Genes, however, may not be translated and instead code for regulatory or structural RNA molecules.

As used herein, the term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

The term “genome” is intended to include the entire DNA complement of an organism, including the nuclear DNA component, chromosomal or extrachromosomal DNA, as well as the cytoplasmic domain (e.g., mitochondrial DNA).

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

The term “label” as used herein refers to any compound or molecule known in the art that can be used to provide a detectable and/or quantifiable effect. The label can be attached to a binding agent. In some embodiments, the label can be attached to a nucleic acid or protein (e.g., antibody, antibody fragment, and/or enzyme). In some embodiments, the label can be attached to a probe system.

The term “nucleotide probe” as used herein refers to a nucleic acid sequence which is complementary to a nucleic acid sequence that is present or enriched in a fetal cell or a maternal cell. In some embodiments, the nucleotide probe is an RNA, a DNA, a GNA or an LNA probe.

The term “polynucleotide” as used herein refers to a sequence of nucleotides connected by phosphodiester linkages. Polynucleotides are presented herein in the direction from the 5′ to the 3′ direction. A polynucleotide can be a deoxyribonucleic acid (DNA) molecule or ribonucleic acid (RNA) molecule. Where a polynucleotide is a DNA molecule, that molecule can be a gene or a cDNA molecule. Nucleotide bases are indicated herein by a single letter code: adenine (A), guanine (G), thymine (T), cytosine (C), inosine (I) and uracil (U). A polynucleotide can be prepared using standard techniques well known to one of skill in the art.

According to the present disclosure, any amino acid-based molecule (natural or non-natural) may be termed a “polypeptide” and this term embraces “peptides,” “peptidomimetics,” and “proteins.” As used herein, “polypeptide” means a polymer of amino acid residues (natural or unnatural) linked together most often by peptide bonds. The term, as used herein, refers to proteins, polypeptides, and peptides of any size, structure, or function. A “peptidomimetic” or “polypeptide mimetic” is a polypeptide in which the molecule contains structural elements that are not found in natural polypeptides (i.e., polypeptides comprised of only the 20 proteinogenic amino acids). In some embodiments, peptidomimetics are capable of recapitulating or mimicking the biological action(s) of a natural peptide.

As used herein, the term “sample” refers to an aliquot or portion taken from a source and/or provided for analysis or processing. In some embodiments, a sample is from a biological source such as a tissue, cell or component part (e.g., a body fluid, including but not limited to blood, vaginal fluid, mucus, lymphatic fluid, synovial fluid, cerebrospinal fluid, saliva, amniotic fluid, amniotic cord blood, urine, and semen), also termed as a biological sample. In some embodiments, a sample may be or include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, or organs. In some embodiments, a sample is or includes a medium, such as a nutrient broth or gel, which may contain cellular components, such as proteins. In some embodiments, a “primary” sample is an aliquot of the source. In some embodiments, a primary sample is subjected to one or more processing (e.g., separation, purification, etc.) steps to prepare a sample for analysis or other use.

The term “sheath fluid” as used herein refers to a carrier fluid that runs in a microfluidic cell separator. In some embodiments, a suspension of fetal cells can be injected into the center of a flowing sheath fluid.

The terms “sufficient” and “effective”, as used interchangeably herein, refer to an amount (e.g., mass, volume, dosage, concentration, and/or time period) needed to achieve one or more desired result(s).

As used herein, the term “trophoblast” refers to an epithelial cell which is derived from the placenta of a mammalian embryo or fetus. A trophoblast typically contacts the uterine wall. Generally, three types of trophoblasts are present, the villous cytotrophoblast, the syncytiotrophoblast and the extravillous trophoblast.

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety.

### EXAMPLES

### Example 1: Enrichment of Fetal Trophoblasts Using Namocell™

The Namocell™ instrument was turned on and the trigger was changed to fluorescein isothiocyanate (FITC) channel. The system was initialized by following the instructions on screen. The cleaning cartridge was loaded, water was removed from the sheath bottle, and PBS was added to a sheath bottle. A collection tube was added to the waste bottle to collect sample flowthrough. A CyteSlide™ was prepared by adding PBS and DAPI to each well. The cells were thoroughly and gently resuspended until homogeneous cell suspension is achieved. Then cell suspension was added to the cell cartridge. The cartridge was quickly loaded into holder and instructions are followed on screen. The cell cartridge was aligned using metal knob to center the laser spot between the walls of the channel. The enrich mode was clicked and a CyteSlide™ was placed on the stage in upper left corner. If the depositing rate was too fast or too slow, the FITCL is adjusted prior to enrichment of cells. The sample was allowed to enrich into the main wells of the CyteSlide™. Fetal trophoblasts were stained with antibodies to cytokeratin and detected in the FITC channel of the Namocell™ instrument. Maternal cells were stained with antibodies to CD45 and detected in the PE channel of the Namocell™ instrument. This process of enrichment deposited a droplet containing 500-1000 maternal cells along with one FITC+ event that triggered the deposit to occur. A small subset of these deposits contained a true fetal cell. This process achieved approximately 100-fold enrichment of fetal trophoblasts from maternal white blood cells, decreasing the maternal cell count from about 200 million to 500,000-2 million. When entire sample had been enriched, the collection tube was removed from the waste bottle, sealed with parafilm, and stored at 2-8° C. until cell yield was determined. Once the CyteSlide™ had cells enriched into both wells, the cells were centrifuged and RareCyte™ scanning was performed. If fewer than 4 fetal trophoblasts are identified in the initial Namocell™ enrichment, this protocol may be repeated using the flowthrough fraction. If fewer than 4 fetal trophoblasts are identified after the second round of Namocell™ enrichment, the sample may be further enriched by FACS.

### Example 2: Enrichment of Fetal Trophoblasts Using Namocell™ Followed by FACS

The fetal cells were enriched by Namocell™ as shown in Example 1. The sample was further enriched by FACS, if fewer than 4 fetal trophoblasts were identified after the second round of Namocell™ enrichment. The system was initialized by following the startup procedure. After startup was complete, the cells were resuspended in PBS and the cell suspension was added to a FACS tube. The sample was loaded, PMT voltages were adjusted on the SSC/FSC plot as well as the PE/A488 plot until the groups of cells fell into the designated gates. In summary, the gate on the SSC/FSC plot encompassed the large mass of cells in the center, the rectangular gate on the PE/A488 encompassed most of the maternal cells (which should be PE+ and A488−), and the large CK+ (cytokeratin) gate encompassed most of the cells which exhibited high A488 signal (some of which are fetal trophoblast cells). CK+ cells were sorted into a CyteSlide™. When the sample in the FACS tube was consumed, the sorting was stopped, more sample was added, and the process of sorting CK+ cells into a CyteSlide™ was repeated until entire sample was consumed. The sample was centrifuged and scanned on the RareCyte CyteFinder™ to identify any additional fetal trophoblasts.

### Example 3: Detection of Fetal Trophoblasts Using Rarecyte™ Scanning

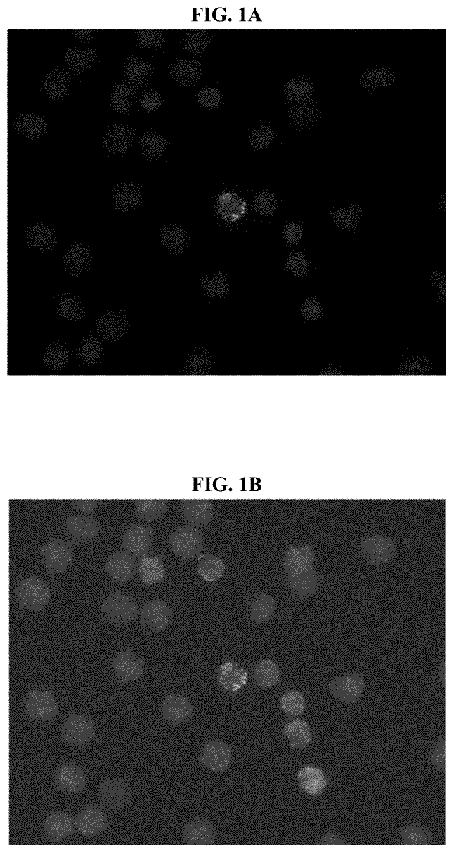

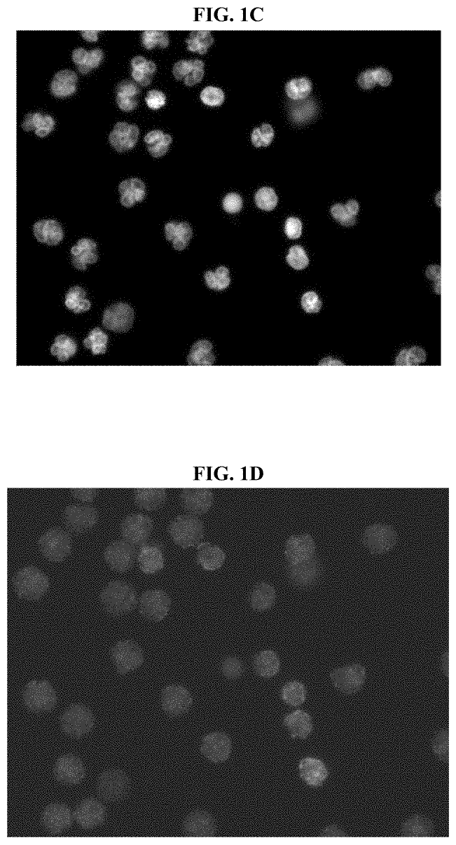

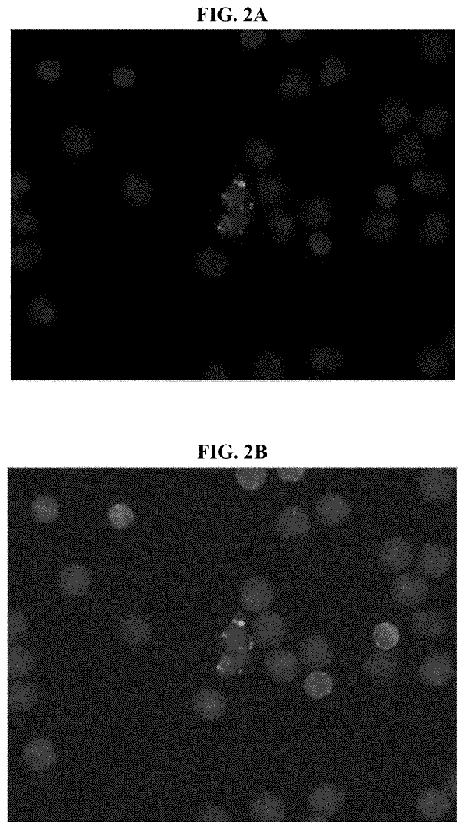

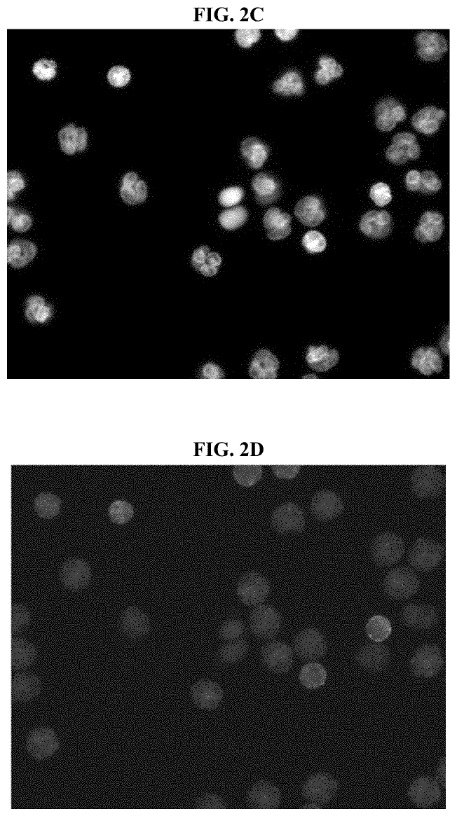

The enriched cells were scanned by selecting the NIPT scanning method. The cell nuclei (stained with DAPI) were used for focusing and the sample was interrogated for any cells containing a nucleus plus cytokeratin (stained with Alexa Fluor 488) or a nucleus without CD45 (stained with PE). This method also identified debris in a fourth far-red channel. The slide was inserted into the RareCyte CyteFinder™ to analyze the images. When the analysis was complete, the gallery of images were identified to determine which were true trophoblasts. Candidate trophoblasts were microscopically verified by examination at 40× magnification. Images of trophoblasts were recorded, and coordinates were saved for cell picking. FIG. 1A-1D are images showing a singlet trophoblast, maternal cells and cell nuclei in various channels used for identification. In FIG. 1A, the blue color shows cell nuclei and the green color shows singlet fetal trophoblast with cytokeratin as the cell marker. FIG. 1B is an image showing overlay of all channels. The blue color shows cell nuclei, the green color shows singlet fetal trophoblast with cytokeratin as the cell marker and the yellow color shows maternal cells with CD45 as the cell marker. FIG. 1C is an image showing cell nuclei. FIG. 1D is an image showing maternal cells. The blue color shows cell nuclei and the yellow color shows maternal cells with CD45 as the cell marker. Similarly, FIG. 2A-2D are images showing a doublet trophoblast, maternal cells and cell nuclei in various channels used for identification.

### Example 4: Isolation of Fetal Trophoblasts Using Rarecyte™ Picking

A fresh needle was loaded and calibrated. Coordinates for desired sample were loaded into the coordinates window. Repicking wells of sample CyteSlide™ were coated with 2% BSA in PBS, then repicking wells were filled with fresh PBS. The slide was loaded into the machine. The needle was flushed and filled with PBS. The position of the first cell to be picked was navigated. The needle was lowered, the cell was aspirated, and the needle was raised. Navigate to the repicking well and the needle was lowered. Small volumes from the needle were expelled until the target cell comes out. The target cell was followed until it stops moving and its position was saved in the coordinates window. The needle was flushed, and the process was repeated with all other cells to be picked from the sample. When all fetal cells were in the repicking well, the needle was flushed thoroughly and wiped down gently with 70% ethanol on a Kimwipe. Old PBS in the flush and fill tubes were replaced with fresh PBS. An optical-bottom CytePicker™ tube was inserted in the depositing position. The needle was filled with PBS and navigated to the first fetal cell to be picked. The needle was lowered, the fetal cell was aspirated, and the needle was raised. Navigate to the CytePicker™ tube and the cell was deposited into the tube. The tube was removed from the machine and labelled it with the sample ID and cell number. The steps were repeated with all other fetal cells to be picked. Each cell was deposited in its own tube. The slides were discarded when all target cells were picked.

### Example 5: Detection of Male and Female Cells Using RNAScope™

The target cell population to be processed was collected via RBC lysis, density gradient, buffy coat collection. Namocell™, MACS, FACS, or any other cell sorting method designed to enrich for a specific cell population. Alternatively, the cultured cells were harvested. The cells were fixed in 4% PFA, then the supernatant was washed and removed. The cells were permeabilized in 1× perm solution, then supernatant was washed and removed. The slides were coated in poly-L lysine. The cells were resuspended in 70% EtOH and cell suspension was applied to slides. The cell suspension was allowed to dry and hydrophobic barrier was drawn around the cell spot. The cells were dehydrated in 50% EtOH, then 70% EtOH, 100% EtOH, and again in 100% EtOH. The slides were allowed to dry, then rinsed with PBS. Probes were applied to the cell spot(s). In this case, probes of one channel were targeted for male-specific RNA molecules (TTTY15, UTY, KDM5D, DDX3Y, EIF1AY, ZFY, TMSB4Y, USP9Y, RPS4Y1, NLGN4Y, or any combination of multiple such male-specific probes). Another channel targeted female-specific RNA molecules, XIST. For the male-specific probes, multiple probes may be used individually, in combination, or in different channels. The cells were incubated at 40° C. for 2 hours. The cells were washed twice, and the first amplification solution (AMP1) was applied. This is an oligonucleotide that binds to the tail sequence of the probes in the previous step. The cells were incubated at 40° C. for 30 minutes. The cells were washed twice, and the second amplification solution (AMP2) was applied. This is an oligonucleotide that binds to the tail sequence of AMP1 in the previous step. The cells were incubated at 40° C. for 15 minutes. The cells were washed twice, and the third amplification solution (AMP3) was applied. This is an oligonucleotide that binds to the tail sequence of AMP2 in the previous step. The cells were incubated at 40° C. for 30 minutes. The cells were washed twice, and the fourth amplification solution (AMP4) was applied. This is a fluorescently labelled oligonucleotide that binds to the tail sequence of AMP3 in the previous step. There are multiple options for fluorescent combinations corresponding to the three available channels. The cells were incubated at 40° C. for 15 minutes. The cells were washed twice and PBS+DAPI was added. The cells were imaged via automated scanning or manual examination to identify male and female cells. In maternal samples, male fetal cells expressed male RNA markers above normal background levels, and lower amounts of XIST than the average maternal cell. Maternal cells did not express male RNA markers above normal background levels and expressed high levels of XIST RNA.

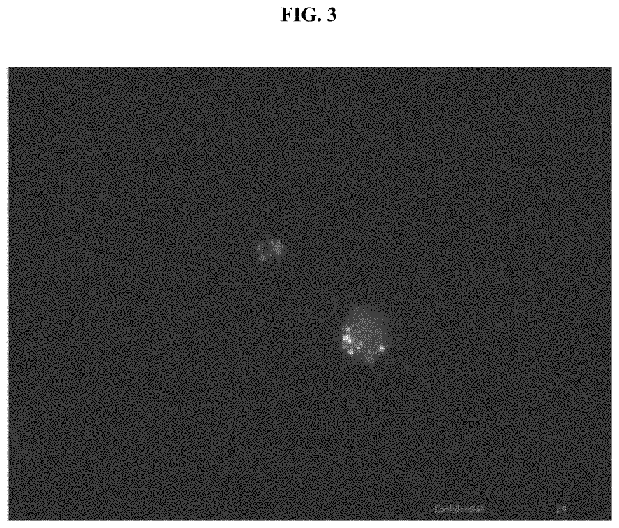

FIG. 3 is an image showing distinct staining of male cells compared to female cells. In the image, the red color shows the female cell with XIST cell marker and yellow color shows the male cell with TTTY15 cell marker.

### Example 6: Genotyping to Distinguish Fetal Cells From Maternal Cells

Low coverage SNP data from each cell acquired by next-generation sequencing was compared against the results of a high-density SNP array performed on the mother's genomic DNA. As shown in FIG. 4, cells were classified based on comparison of maternal vs. fetal SNP data and established thresholds. A fetal cell has hundreds to thousands of alleles which are not present in the mother as these alleles are inherited from the father, hence classifying it as fetal and not maternal.

### Example 7: Single Trophoblast Cell Sorting by FACS

Red blood cells were removed, and the remaining white blood cells were fluorescently labeled with CD45-PE, CD45-AlexaFluor647, and cytokeratin-AlexaFluor488 antibodies. Fetal trophoblast cells labeled with cytokeratin-AlexaFluor488 were enriched on the Namocell™ instrument as described in Example 1 but were deposited into a clean microcentrifuge tube instead of a CyteSlide™. The nuclei of cells in the enriched cell fraction were stained with Hoechst 33342 and loaded into a FACS tube. The system was initialized by following the startup procedure. The sample was loaded, PMT voltages were adjusted on the SSC/FSC plot, PE/A488 plot, and A647/FSC plots until the groups of cells fell into the designated gates. In summary, the gate on the SSC/FSC plot encompassed the lymphocytes and monocytes, the rectangular gate on the Hoechst/FSC plot encompassed the nucleated cells, the rectangular gate on the PE/A488 plot encompassed most of the maternal cells (PE+ and A488−), the CK+ (cytokeratin)/CD45-gate encompassed most of the cells which exhibited high A488 signal and low CD45-PE signal (some of which are fetal trophoblast cells), the rectangular gate on the A647/FSC plot encompassed the fetal trophoblast cells from within the CK+ gate (maternal cells labeled with CD45-AlexaFluor647 were excluded), and the gate on the final SSC/FSC plot designated cells which were ultimately sorted as candidate trophoblasts. Cells were individually sorted into wells of a 96- or 384-well plate. After sorting, cells were subjected to whole genome amplification, library preparation, and next generation sequencing. Sorted cells were identified as fetal by genotyping as described in Example 6. The sorting process typically yielded 5-15 sorted cells from the starting number of 1-2 million cells in the enriched fraction, of which approximately 50% were fetal trophoblasts.

### Equivalents and Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A method of isolating fetal cells from a biological sample from a pregnant subject, said method comprising the steps of:
(a) contacting the biological sample with one or more binding agents directed against at least one of the group consisting of a fetal cell marker, a maternal cell marker, and a nuclear marker;
(b) sorting the fetal cells based on detection of the one or more binding agents bound to the nuclear marker, the fetal cell marker, or the maternal cell marker, wherein the sorting is performed using a microfluidic cell separator, microbubbles-based cell sorting device, fluorescence activated cell sorting (FACS) device, or magnetic activated cell sorting (MACS) device; and
(c) determining the presence of the fetal cells as those bound by the one or more binding agents from step (b).

2. (canceled)

3. The method of claim 1, wherein the biological sample is obtained at a gestational age of less than about 17 weeks.

4. (canceled)

5. The method of claim 1, wherein the one or more binding agents is a nucleotide probe, or an antibody or a fragment thereof.

6. The method of claim 5, wherein the nucleotide probe is an RNA probe, a DNA probe, a GNA probe,. or an LNA probe.

7-20. (canceled)

21. The method of claim 1, step (b) further comprises a step of isolating the fetal cells using a single-cell picking device.

22. (canceled)

23. The method of claim 1, wherein the fetal cell marker is a fetal cell nucleic acid marker, a fetal cell cytoplasmic marker, a fetal cell nuclear marker, a fetal cell surface marker, a fetal epithelial cell marker, or a fetal endothelial cell marker.

24. The method of claim 23, wherein the fetal cell nucleic acid marker is XIST, TTTY15, RPS4Y1, KRT7, EPCAM, HLA-G, ENG, or βHCG.

25-27. (canceled)

28. The method of claim 23, wherein the fetal epithelial cell marker is cytokeratin.

29-31. (canceled)

32. The method of claim 23, wherein the fetal endothelial cell marker is Endoglin/CD105.

33. (canceled)

34. The method of claim 1, wherein the maternal cell marker is CD45.

35. (canceled)

36. The method of claim 1, wherein the fetal cells are fetal trophoblast cells.

37-38. (canceled)

39. The method of claim 1, wherein the biological sample is a blood sample or a cervical secretion sample from the pregnant subject.

40. The method of claim 1, wherein the biological sample is further enriched for the fetal cells.

41. The method of claim 1, wherein the one or more binding agents is covalently or non-covalently bound to a label.

42. (canceled)

43. The method of claim 41, wherein the label is a dye, a fluorescent dye, a radiolabel, a hapten, a luminogenic, a phosphorescent or a fluorogenic moiety, or a mass tag.

44-46. (canceled)

47. A method of detecting fetal cells in a biological sample from a pregnant subject, said method comprising the steps of:
(a) contacting the biological sample with one or more binding agents directed against at least one of the group consisting of a nuclear marker, a fetal cell marker, or a maternal cell marker;
(b) visualizing the fetal cells based on detection of the one or more binding agents bound to the nuclear marker, the fetal cell marker, or the maternal cell marker; and
(c) detecting the presence of the fetal cells as those bound by the one or more binding agents from step (b).

48. The method of claim 47, wherein the visualizing in step (b) is performed under a microscope or a laser.

49. (canceled)

50. The method of claim 47, wherein the biological sample is obtained at a gestational age of less than about 17 weeks.

51. (canceled)

52. The method of claim 47, wherein the one or more binding agents is a nucleotide probe, or an antibody or a fragment thereof.

53. The method of claim 52, wherein the nucleotide probe is an RNA probe, a DNA probe, a GNA probe, or an LNA probe.

54-61. (canceled)

62. The method of claim 47, wherein the fetal cell marker is a fetal cell nucleic acid marker, a fetal cell cytoplasmic marker, a fetal cell nuclear marker, a fetal cell surface marker, a fetal epithelial marker, or a fetal endothelial cell marker.

63. The method of claim 62, wherein the fetal cell nucleic acid marker is XIST, TTTY15, RPS4Y1, KRT7, EPCAM, HLA-G, ENG, or βHCG.

64-66. (canceled)

67. The method of claim 62, wherein the fetal epithelial cell marker is cytokeratin.

68-70. (canceled)

71. The method of claim 62, wherein the fetal endothelial cell marker is Endoglin/CD105.

72. (canceled)

73. The method of claim 47, wherein the maternal cell marker is CD45.

74. (canceled)

75. The method of claim 47, wherein the fetal cells are fetal trophoblast cells.

76-77. (canceled)

78. The method of claim 47, wherein the biological sample is a blood sample or a cervical secretion sample from the pregnant subject.

79. The method of claim 47, wherein the biological sample is further enriched for the fetal cells.

80. The method of claim 47, wherein the one or more binding agents is covalently or non-covalently bound to a label.

81. (canceled)

82. The method of claim 80, wherein the label is a dye, a fluorescent dye, a radiolabel, a hapten, a luminogenic, a phosphorescent or a fluorogenic moiety, or a mass tag.

83. (canceled)

84. The method of claim 82, wherein the fluorescent dye is a xanthene dye, a coumarin dye, a pyrene dye, or a cyanine dye.

85. (canceled)

86. A method for genotyping a fetus, the method comprising:
(a) isolating the fetal cells according to claim 1;
(b) lysing the fetal cells to obtain the fetal nucleic acids;
(c) amplifying the fetal cell nucleic acids or a portion thereof; and
(d) genotyping the fetus by evaluating for a genetic difference between the fetus and the pregnant subject.

87. The method of claim 86, wherein the genetic difference is a copy number variation of a gene or a chromosomal region, a translocation, a polymorphism, an indel, a single nucleotide polymorphism (SNP), mosaicism, confined placental mosaicism, uniparental disomy, or a nucleic acid sequence or an allele associated with a pathological condition.

88-101. (canceled)

102. The method of claim 86, wherein step (c) comprises whole genome amplification.

103. The method of claim 86, wherein step (d) comprises quantitative polymerase chain reaction amplification (qPCR), SNP array, array comparative genomic hybridization (array CGH), next generation sequencing (NGS), single cell NGS, or Short Tandem Repeat analysis (STR analysis).

104. (canceled)


SEQUENCE LISTING (1 sequence)

SEQ ID NO: 1 - protein, 263 aa, Homo sapiens
  REGION 1..263: note = ENSG00000129824.16 Ribosomal Protein S4 Y-linked 1 (RPS4Y1)
MARGPKKHLKRVAAPKHWMLDKLTGVFAPRPSTGPHKLRECLPLIVFLRNRLKYALTGDEVKKICMQRFIKIDGKVRVDVTYPAGFMDVISIEKTGEHFRLVYDTKGRFAVHRITVEEAKYKLCKVRKITVGVKGIPHLVTHDARTIRYPDPVIKVNDTVQIDLGTGKIINFIKFDTGNLCMVIGGANLGRVGVITNRERHPGSFDVVHVKDANGNSFATRLSNIFVIGNGNKPWISLPRGKGIRLTVAEERDKRLATKQSSG